(19) United States
(12) Patent Application Publication
(10) Pub. No.: US 2025/0025322 A1
(43) Pub. Date: Jan. 23, 2025

(54) GRAFT DEPLOYMENT AND ATTACHMENT SYSTEMS AND METHODS

(71) Applicant: ACTIVAS Orthopedics, Inc., Coronado, CA (US)

(72) Inventors: Colin Murphy, Carlsbad, CA (US); Benjamin Arnold, San Diego, CA (US); Brian Bowman, Carlsbad, CA (US); Mark H. Getelman, Tarzana, CA (US); Thomas A. Afzal, Coronado, CA (US)

(21) Appl. No.: 18/780,209

(22) Filed: Jul. 22, 2024

Related U.S. Application Data
(60) Provisional application No. 63/528,144, filed on Jul. 21, 2023.

(51) Int. Cl. A61F 2/954; A61F 2/95; A61F 2/966

(52) U.S. Cl. CPC A61F 2/954; A61F 2/9517; A61F 2002/9665

(57) ABSTRACT

Systems, devices, and methods for deploying a graft to a treatment site of a patient. Exemplary systems include a device body and an insertion mechanism. The device body can have a handle and a trigger. The insertion mechanism can be in operative association with the device body, and can have an outer sheath, a support shaft disposed at least partially within the outer sheath, an expansion mechanism coupled with the support shaft, and a graft in contact with the expansion mechanism.

### CROSS-REFERENCES TO RELATED APPLICATIONS

This application claims the benefit of U.S. Provisional Patent Application No. 63/528,144 filed Jul. 21, 2023, the disclosure of which is incorporated herein by reference.

### BACKGROUND

Embodiments of the present invention relate generally to graft deployment systems and methods, and in particular instances, to graft deployment systems and methods for rotator cuff repair and reconstruction.

Current graft deployment modalities enable surgeons to provide beneficial treatments to patients in need thereof. Yet still further improvements in graft deployment technology are desired. Embodiments of the present invention provide solutions to at least some of these outstanding needs.

### BRIEF SUMMARY

Exemplary graft deployment and attachment systems as disclosed herein can be provided as a single use device for the deployment and attachment of graft for rotator cuff reconstruction or rotator cuff repair. The deployment device can include a handle, a control such as a trigger, and an insertion mechanism. The insertion mechanism may include an outer sheath, a central shaft, and an expansion mechanism. The insertion mechanism can come preloaded with graft.

The system may additionally comprise medial graft fixation devices (anchors) and medial graft fixation deployment devices. A suture loading device may be provided for shaping a graft in a sinusoidal configuration and facilitating weaving a suture through the graft. Lateral anchors may also be provided. An expandable spacer may be provided such as in the form of a resorbable container filled with a gel interior. A loading method and device may be provided for loading a lateral anchor with a barbed suture.

The system may additionally comprise a spacer, such as for deployment into the subacromial space of a shoulder joint. The spacer may include a biodegradable outer barrier, enclosing a gel such as a hydrogel, bioactive glass or other absorbable material, preferably capable of expansion upon contact with saline or other fluid such as a body fluid.

To deploy the graft the control trigger can be used to draw back the sheath and then expand the expansion mechanism, which presses the graft into position. Anchors may then be deployed. The expansion mechanism can then be collapsed, and the insertion mechanism can be removed, leaving the graft in position for fixation.

In one aspect, embodiments of the present invention encompass graft deployment devices and related methods. Exemplary devices include a device body and an insertion mechanism in operative association therewith, the insertion mechanism carrying an expandable or deployable graft. A method of delivering a graft to a treatment site of a patient can include positioning a distal portion of a graft deployment system at the treatment site of the patient, and actuating the graft deployment device to facilitate expansion of the graft.

In another aspect, embodiments of the present invention encompass graft deployment devices and related methods of use. An exemplary graft deployment device can include a device body and an insertion mechanism. The device body can include a handle and a trigger. The insertion mechanism can be in operative association with the device body, and can include an outer sheath, a support shaft disposed at least partially within the outer sheath, and an expansion mechanism coupled with the support shaft. The expansion mechanism can be configured to support a graft. In some embodiments, the expansion mechanism includes a plurality of wires. Individual wires can each include a distal end configured to puncture the graft. In some embodiments, the plurality of wires can be configured to expand the graft when the plurality of wires are extended in a distal direction through the support shaft. In some cases, each of the plurality of wires includes a stop disposed proximal to the distal end, and the stop is configured to prevent the wire from pushing fully through the graft. In some cases, one or more of the plurality of wires includes nitinol. In some cases, the device body includes a suture routing mechanism configured to receive a suture therethrough. In some cases, the expansion mechanism includes a plurality of articulating arms. In some cases, the handle and trigger are in linear relationship with one another. In some cases, the handle and trigger are in a pistol grip configuration. In some cases, the device body includes a sheath lever configured to retract the outer sheath. In some cases, the outer sheath is spring loaded. In some cases, the device includes the graft.

In another aspect, embodiments of the present invention encompass methods of delivering a graft to a treatment site of a patient. Exemplary methods may include positioning a distal portion of a graft deployment device at the treatment site of the patient. The graft deployment device can include an outer sheath and an expansion mechanism configured to support the graft. Methods can also include actuating a trigger of the graft deployment device to activate the expansion mechanism and expand the graft, and attaching the graft to the treatment site of the patient. In some cases, the expansion mechanism includes a plurality of wires, and each of the plurality of wires includes a distal end configured to puncture the graft. In some cases, the plurality of wires are configured to expand the graft when the plurality of wires are extended in a distal direction through the support shaft. In some cases, each of the plurality of wires includes a stop disposed proximal to the distal end. In some cases, the stop is configured to prevent the wire from pushing fully through the graft. In some cases, each of the plurality of wires includes nitinol. In some cases, the graft is attached with a suture, and the device body includes a suture routing mechanism configured to receive the suture therethrough. In some cases, the expansion mechanism includes a plurality of articulating arms. In some cases, the handle and trigger are in linear relationship with one another. In some cases, the handle and trigger are in a pistol grip configuration. In some cases, methods can include retracting the outer sheath by actuating a sheath lever of the device body.

In another aspect, embodiments of the present invention encompass suture loading block devices for loading a graft with a suture. Exemplary suture loading block devices can include a top body having a first contoured surface, and a bottom body coupled with the top body. The bottom body can include a second contoured surface that is complementary to the first contoured surface of the top body. Suture loading block devices can also include a locking mechanism configured to lock the top body and the bottom body together when the loading block device is in a closed configuration. In some cases, the first contoured surface and the second contoured surface define a graft space that is configured to receive the graft therein when the loading block is in the closed configuration. In some cases, the first contoured surface includes a plurality of first ridge troughs, the second contoured surface includes a plurality of second ridge troughs, and the plurality of first ridge troughs and the plurality of second ridge troughs provide a passageway that is configured to receive a needle loaded with the suture therethrough when the loading block device is in the closed configuration. In some cases, the bottom body includes a cannula that is configured to be in coaxial alignment with the passageway when the loading block device is in the closed configuration. In some cases, the first contoured surface includes a series of first ridges and a series of first valleys, and the second contoured surface includes a series of second ridges and a series of second valleys. In some cases, the graft space has a sinusoidal configuration. In some cases, the first contoured surface includes a plurality of third ridge troughs, the second contoured surface comprises a plurality of fourth ridge troughs, and the plurality of third ridge troughs and the plurality of fourth ridge troughs provide a second passageway that is configured to receive a second needle loaded with a second suture therethrough when the loading block device is in the closed configuration. In some cases, the top body includes a top transverse aperture, and the bottom body includes a bottom transverse aperture, and the top transverse aperture and the bottom transverse aperture provide a transverse passageway that is oriented perpendicular to the graft space. In some cases, the needle is attached with a removable handle. In some cases, the needle is attached with a needle passer. In some cases, the suture loading block device includes a needle cap configured for removeable attachment with a distal end of the needle. In some cases, the locking mechanism includes a lever. In some cases, the locking mechanism is configured for automatic engagement with a spring load when the suture loading block device is in the closed configuration, and the locking mechanism is configured to be released by actuation of a button. In some cases, the suture loading block device is spring loaded to open when the locking mechanism is released.

In yet another aspect, embodiments of the present invention encompass methods of loading a graft with a suture. Exemplary methods can include placing the graft on a bottom body of a suture loading block device, and closing a top body of the suture loading block device onto the bottom body, so that the suture loading block device is in a closed configuration and the graft is disposed in a graft space defined by the bottom body and the top body. Methods can also include passing a needle loaded with the graft through a passageway defined by the top body and the bottom body, so that the suture is loaded on the graft. In some cases, the top body has a first contoured surface, the bottom body has a second contoured surface that is complementary to the first contoured surface of the top body, and the first contoured surface and the second contoured surface define the graft space that is configured to receive the graft therein when the loading block device is in the closed configuration. In some cases, the first contoured surface includes a plurality of first ridge troughs, the second contoured surface includes a plurality of second ridge troughs, and the plurality of first ridge troughs and the plurality of second ridge troughs provide the passageway. In some cases, the bottom body includes a cannula that is configured to be in coaxial alignment with the passageway when the loading block device is in the closed configuration. In some cases, the first contoured surface includes a series of first ridges and a series of first valleys, and the second contoured surface includes a series of second ridges and a series of second valleys. In some cases, the graft space has a sinusoidal configuration. In some cases, the first contoured surface includes a plurality of third ridge troughs, the second contoured surface comprises a plurality of fourth ridge troughs, and the plurality of third ridge troughs and the plurality of fourth ridge troughs provide a second passageway that is configured to receive a second needle loaded with a second suture therethrough when the loading block device is in the closed configuration. In some cases, the top body includes a top transverse aperture, and the bottom body includes a bottom transverse aperture, and the top transverse aperture and the bottom transverse aperture provide a transverse passageway that is oriented perpendicular to the graft space. In some cases, methods can include attaching the needle with a removable handle or a needle passer. In some cases, methods can include removably attaching a needle cap to a distal end of the needle.

In still yet another aspect, embodiments of the present invention encompass graft loading block devices and methods of their use for loading a graft onto a graft deployment device. Exemplary graft loading block devices can include a first rigid body, a flexible component attached with the first rigid body, a second rigid body attached with the flexible component, and an adapter docking mechanism configured to attach with an adapter. In some cases, the first rigid body, the flexible component, and the second rigid body are configured to fold the graft into a loading configuration. In some cases, the loading configuration is a curled configuration that can be loaded on the graft deployment device. In some cases, the loading configuration is a curled configuration that can be loaded onto a cylinder of the graft deployment device. In some cases, the first rigid body, the flexible component, and the second rigid body are configured to fold the graft into the loading configuration on a shaft of a graft deployment device. In some cases, the graft loading block device can include the adapter, and the adapter can be configured for removable attachment with the graft deployment device. In some cases, the graft loading block device can include the adapter, and the adapter can be configured for attachment with the graft loading block device and the graft deployment device during loading of the graft onto the graft deployment device. In some cases, the loading configuration is a curled configuration that can be loaded into a sheath of the graft deployment device. In some cases, the loading configuration is a curled configuration that can be loaded into a cannula. In some cases, the first rigid body includes a first locking mechanism, and the second rigid body includes a second locking mechanism that is configured to reversibly lock with the first locking mechanism. In some cases, the first rigid body includes a first locking mechanism, and the second rigid body includes a second locking mechanism that is configured to slidably engage the first locking mechanism.

In another aspect, embodiments of the present invention encompass methods of preparing a graft for loading using a graft loading block device. Exemplary methods can include placing the graft on a flexible component of the graft loading block, the flexible component is attached with a first rigid body of the graft loading block and with a second rigid body of the graft loading block. Methods can also include moving the first rigid body and the second rigid body relative to one another to cause the flexible component to fold the graft into a loading configuration. In some cases, methods can include attaching a graft deployment device with the graft loading block device via an adapter, and loading the folded graft onto the graft deployment device. In some cases, the adapter is configured for removable attachment with the graft deployment device. In some cases, methods can include loading the folded graft onto a shaft of a graft deployment device. In some cases, methods can include loading the folded graft onto a plurality of wires of a graft deployment device. In some cases, each of the plurality of wires includes a respective stop which prevents its respective wire from puncturing through the graft. In some cases, methods can include loading the folded graft into a sheath of a graft deployment device. In some cases, methods can include loading the folded graft into a cannula. In some cases, the first rigid body can include a first locking mechanism and the second rigid body can include a second locking mechanism that is configured to reversibly lock with the first locking mechanism. In some cases, the first rigid body includes a first locking mechanism, and the second rigid body includes a second locking mechanism that is configured to slidably engage the first locking mechanism.

### BRIEF DESCRIPTION OF THE DRAWINGS

Inventive features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates aspects of a graft deployment device, according to embodiments of the present invention;

FIG. 2 illustrates aspects of a graft deployment device, according to embodiments of the present invention;

FIG. 3 illustrates aspects of a graft deployment device, according to embodiments of the present invention;

FIG. 4 illustrates aspects of a graft deployment device, according to embodiments of the present invention;

FIG. 5 illustrates aspects of a graft deployment device, according to embodiments of the present invention;

FIG. 6 illustrates aspects of a graft deployment device, according to embodiments of the present invention;

FIG. 7 illustrates aspects of a graft deployment device, according to embodiments of the present invention;

FIG. 8 illustrates aspects of a graft deployment device, according to embodiments of the present invention;

FIG. 9 illustrates aspects of a graft deployment device, according to embodiments of the present invention;

FIG. 10 illustrates aspects of a graft deployment device, according to embodiments of the present invention;

FIG. 11 illustrates aspects of a graft deployment device, according to embodiments of the present invention;

FIG. 12 illustrates aspects of a graft fixation device, according to embodiments of the present invention;

FIG. 13 illustrates aspects of a graft fixation device, according to embodiments of the present invention;

FIG. 14 illustrates aspects of a graft fixation device, according to embodiments of the present invention;

FIG. 15 illustrates aspects of a graft fixation deployment device, according to embodiments of the present invention;

FIG. 16 illustrates aspects of a graft fixation deployment device, according to embodiments of the present invention;

FIG. 17 illustrates aspects of a graft fixation deployment device, according to embodiments of the present invention;

FIGS. 18A to 18F illustrate aspects of a graft fixation deployment device, according to embodiments of the present invention;

FIGS. 19A to 19D illustrate aspects of a graft fixation deployment device, according to embodiments of the present invention;

FIGS. 20A to 20C illustrate aspects of a suture loading block device, according to embodiments of the present invention;

FIGS. 21A to 21C illustrate aspects of a suture loading block device, according to embodiments of the present invention;

FIGS. 22A to 22D illustrate aspects of a suture loading block device, according to embodiments of the present invention;

FIGS. 23A to 23H illustrate aspects of a suture loading block device, according to embodiments of the present invention;

FIGS. 24A to 24E illustrate aspects of an anchor, according to embodiments of the present invention;

FIGS. 25A and 25B illustrate aspects of an anchor, according to embodiments of the present invention;

FIG. 26 illustrates aspects of an anchor, according to embodiments of the present invention;

FIGS. 27A to 27F illustrate aspects of a spacer, according to embodiments of the present invention;

FIG. 28 illustrates aspects of an anchor loading assembly, according to embodiments of the present invention;

FIGS. 29A to 29E illustrate aspects of a graft and spacer surgical procedure, according to embodiments of the present invention;

FIG. 30 illustrates aspects of a suture device, according to embodiments of the present invention;

FIGS. 31A and 31B illustrate aspects of a suture device, according to embodiments of the present invention;

FIGS. 32A and 32B illustrate aspects of a suture device, according to embodiments of the present invention;

FIGS. 33A and 33B illustrate aspects of a graft deployment device, according to embodiments of the present invention;

FIGS. 34A to 34C illustrate aspects of a graft deployment device, according to embodiments of the present invention;

FIGS. 35A to 35D illustrate aspects of a graft deployment device, according to embodiments of the present invention;

FIGS. 36A to 36G illustrate aspects of a graft deployment device, according to embodiments of the present invention;

FIGS. 37A and 37B illustrate aspects of a graft deployment device, according to embodiments of the present invention;

FIG. 38 illustrates aspects of a fixation deployment device, according to embodiments of the present invention;

FIGS. 39A to 39L illustrate aspects of a fixation deployment device, according to embodiments of the present invention;

FIGS. 40A to 40I illustrate aspects of a spacer deployment device, according to embodiments of the present invention;

FIGS. 41A to 41C illustrate aspects of a graft loading block device, according to embodiments of the present invention; and

FIGS. 42A to 42J illustrate aspects of a graft loading block device and deployment device, according to embodiments of the present invention.

### DETAILED DESCRIPTION

Specific embodiments of the disclosed device, system and method of use will now be described with reference to the drawings. Nothing in this detailed description is intended to imply that any particular component, feature, or step is essential to embodiments of the invention.

Graft deployment systems disclosed herein can be used to deploy grafts to any of a variety of patient treatment sites. In some cases, a graft deployment system can be used to deploy a graft to a rotator cuff of a patient. In some cases, a graft deployment system can be used to deploy a graft for an Achilles tendon repair, a patella tendon repair, a triceps repair, a quadriceps repair, or the like.

Graft deployment systems disclosed herein can be provided with a variety of different expansion mechanisms which operate to facilitate expansion of a graft. Advantageously, embodiments of the present invention enable the performance of simplified surgical procedures by packaging full functionality into a single-use instrument, reducing cost associated with reprocessing. In some cases, preloading of the graft with the deployment tool can remove the need for manual preparation by the surgeon. The expansion feature can maintain rigidity after expansion, allowing for compression of the graft against target tissues, which is difficult with currently known solutions. Exemplary device embodiments simply procedures compared to some known solutions by minimizing the need for suturing.

Moreover, graft deployment systems disclosed herein do not require that a graft be prepared separately from the deployment system and then loaded by the physician into the deployment system. For example, the system can be provided for use by the physician or surgeon (or other individual) in a pre-loaded configuration, where the graft and the system are provided as a combination. Further, embodiments disclosed herein provide solutions where the system makes substantial contact with the graft during deployment, going beyond contact which is limited to edge contact or point contact. Further, embodiments disclosed herein provide solutions that do not require a frame around the edge of a graft for deployment, or that do not require the use of expanding point contacts, and hence can provide enhanced control of the center of a graft during deployment.

Still further, embodiments disclosed herein can be configured with an expansion mechanism which is semi-rigid, thus enabling the surgeon to have the ability to apply selective pressure to the graft during the fixation process, and also giving flexibility to the surgeon in terms of how they place fixation features. What is more, with pre-loaded graft system embodiments, the surgeon or user is not required to load the graft to the system during the operating procedure, thus enabling the surgeon to perform a more efficient procedure. Relatedly, with pre-loaded graft system embodiments, the surgeon or user does not need to perform the step of sizing the graft while performing the procedure. Relatedly, the graft sizing can be handled in the pre-operative period. This advantage can enable the surgeon to perform the surgery in a shorter amount of time, because they are not required to prepare the graft during the procedure. In another advantage, embodiments of the present invention encompass the use of an insertion mechanism which allows for the positioning of the graft with visualization of its depth and location prior to full deployment. What is more, embodiments of the present invention employ the use of expansion mechanisms having a curved shape or profile.

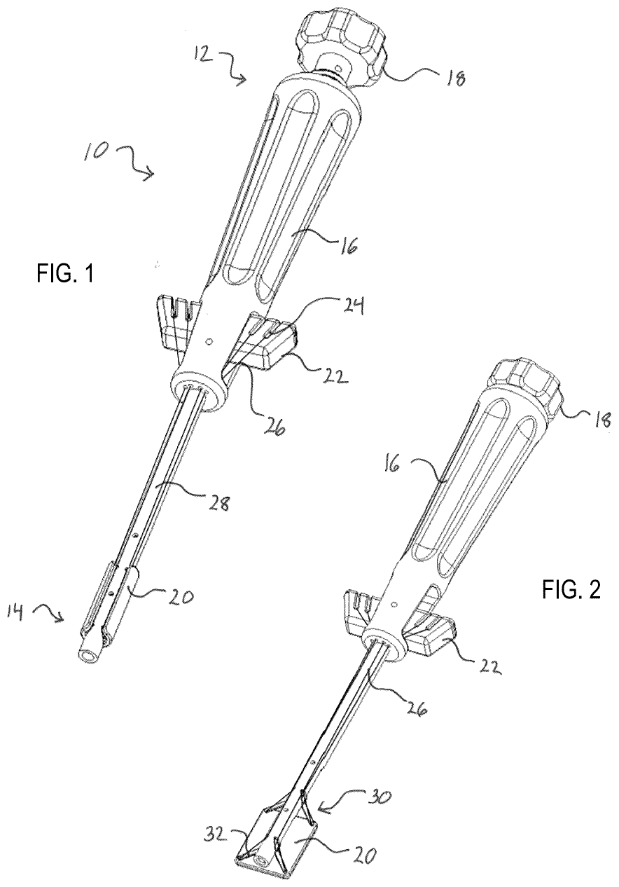

Referring to FIG. 1, there is illustrated a graft deployment device 10 having a proximal end 12 and a distal end 14. A handle 16 of the device supports a graft deployment control 18, which may be a lever, rotatable knob, slider, switch, trigger, or other control. In some embodiments, the handle and graft deployment control can comprise a body of the device. The distal end 14 carries a graft 20 illustrated in FIG. 1 in a low profile (e.g. initial) configuration for advancement to the treatment site, and in FIG. 2 an expanded configuration for implantation and attachment of the graft at the treatment site.

The graft deployment device 10 additionally includes a suture support 22 having a plurality of suture locks or suture locking features or mechanisms 24. In the illustrated implementation, four suture locks 24 are provided. The suture lock may be a post, slot, jam cleat, or other structure for releasably locking a suture. Each suture lock 24 secures the proximal end of a suture 26, which extends distally along an outer shaft 28 of the device to secure the graft 20 to the distal end 14 of the graft deployment device 10. In some embodiments, at least two or three or four or more sutures 26 are utilized to secure the graft 20. In some embodiments, a graft can be secured directly to the arms (e.g. support arms 32) without the assistance of a suture. In the illustrated implementation, four proximal suture ends attached to the suture support may be the proximal ends of four separate sutures the distal ends of which are attached to the graft 20. Alternatively, two sutures can extend distally and pass through and wrap around the distal surface of the graft 20.

Referring to FIG. 2, the graft 20 has been expanded from a rolled or folded low profile configuration (e.g. as depicted in FIG. 1) to a substantially planar expanded configuration. This can be accomplished by actuating a graft support 30 which in some embodiments may include at least one and, in some cases, at least two or four support arms 32. The 20 graft can be removably connected to the graft deployment device 10 by the sutures 26. FIG. 1 depicts the graft deployment control 18 and the suture support 22 in a proximal position, and FIG. 2 depicts the deployment control 18 and the suture support 22 in a proximal position. In some embodiments, graft deployment control 18 is mechanically coupled with suture support 22.

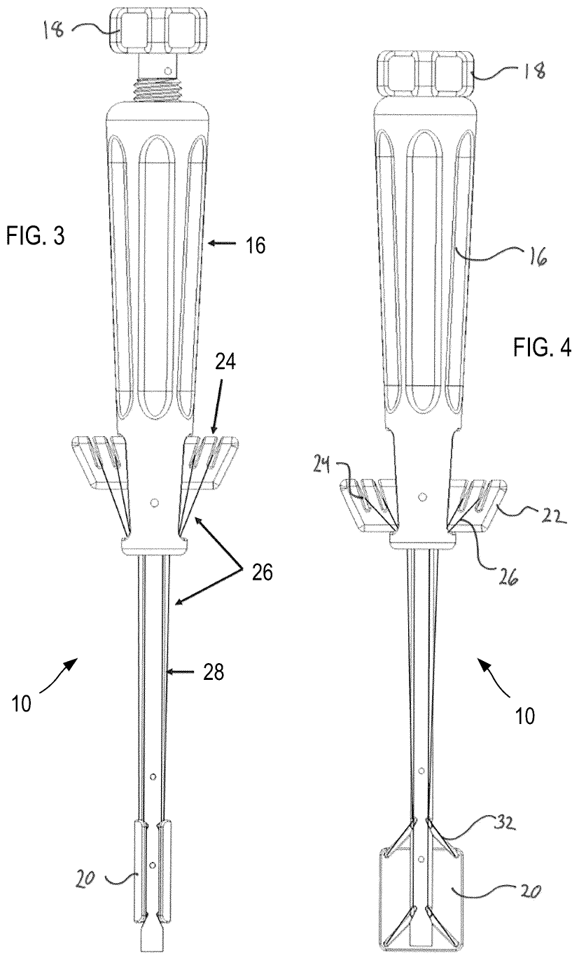

As seen in FIGS. 3 and 4, the control 18 is advanceable from a first position (FIG. 3) in which the graft 20 is in a low profile configuration, to a second position (FIG. 4) in which the graft 20 is in the expanded configuration for attachment to tissue and deployment from the graft deployment device 10.

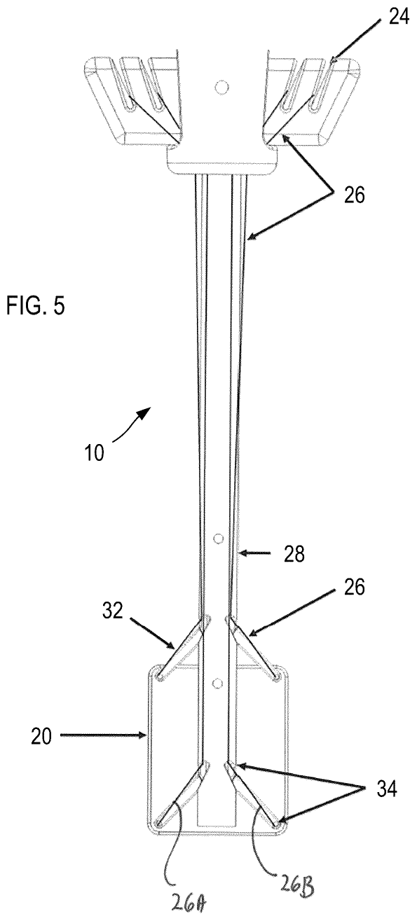

Referring to FIG. 5, each suture 26 extends axially in a distal direction from the suture lock 24 along the outer shaft 28 and is secured to the graft 20. In the illustrated implementation, each suture 26 extends along an arm 32 of the device and secures the graft 20 to the arm 32. Each arm may be provided with one or more suture guides 34 such as an aperture, post, hook, or other guiding structure. In some embodiments, a suture guide 34 can be referred to as a suture routing feature or mechanism. A suture segment 26A and 26B may be two distinct sutures or may be two proximal extensions of a single suture, which loops around the back (distal) side of the graft 20. In some cases, a support arm 26 can be provided as an articulating arm. In some cases, a plurality of arms can operate as an expansion mechanism or as a part of an expansion mechanism.

The suture locks 24 may be spaced apart, such as in a transverse direction to facilitate manual locking and unlocking of each suture one at a time.

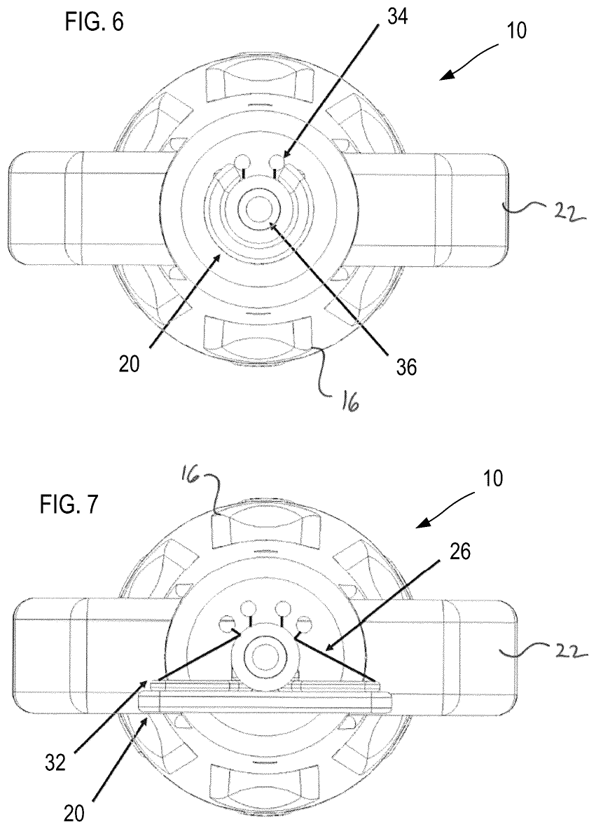

Referring to FIGS. 6 and 7, there are illustrated distal end views of the graft deployment device 10. Arms 32 may be advanced from the low profile configurations shown in FIG. 6, to the expanded configurations shown in FIG. 7 by manipulation of the control 18, which may cause axial displacement such as distal displacement of a central shaft 36 with respect to the outer shaft 28.

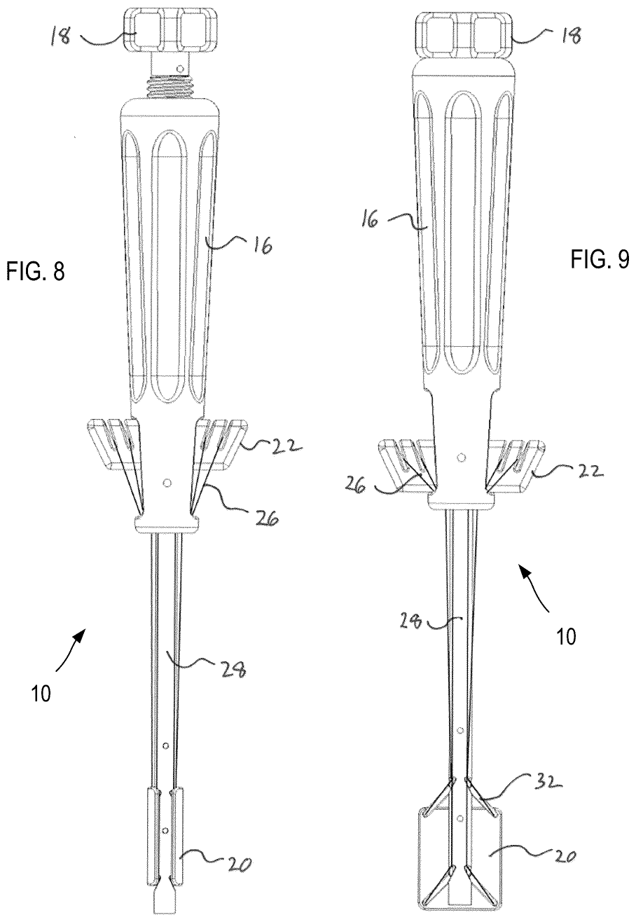

FIGS. 8 to 11 show additional details of the graft deployment device 10, according to embodiments of the present invention. As shown in FIG. 8, a graft deployment device 10 can be inserted, for example to a treatment site of a patient, while in a retracted or initial state. Once in position, the control or knob 18 can be actuated, for example by a user, so as to expand the arms 32 of the device, as depicted in FIG. 9. In some embodiments, tension in the sutures 26 is maintained during the expansion. The device 10 can be used to adjust the position of the graft 20 as desired, for example to a desired position at the treatment location.

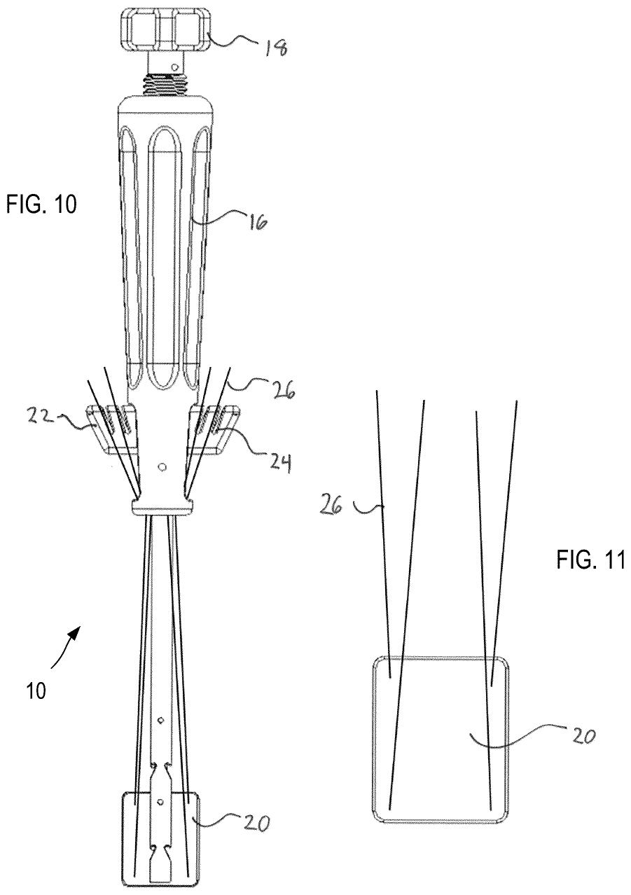

As shown in FIGS. 10 and 11, once the graft 20 is fixated to a desired location in the patient, the sutures 26 can be removed from the suture locking features 24 of the device 10. In some cases, the sutures can be removed from the graft, for example by pulling them from the graft. In some cases, the sutures can be left in place, as attached with the graft. Subsequently, the control or knob 18 can be actuated to retract the arms, and the device 10 can be removed or withdrawn from the patient, leaving the graft 20 in place.

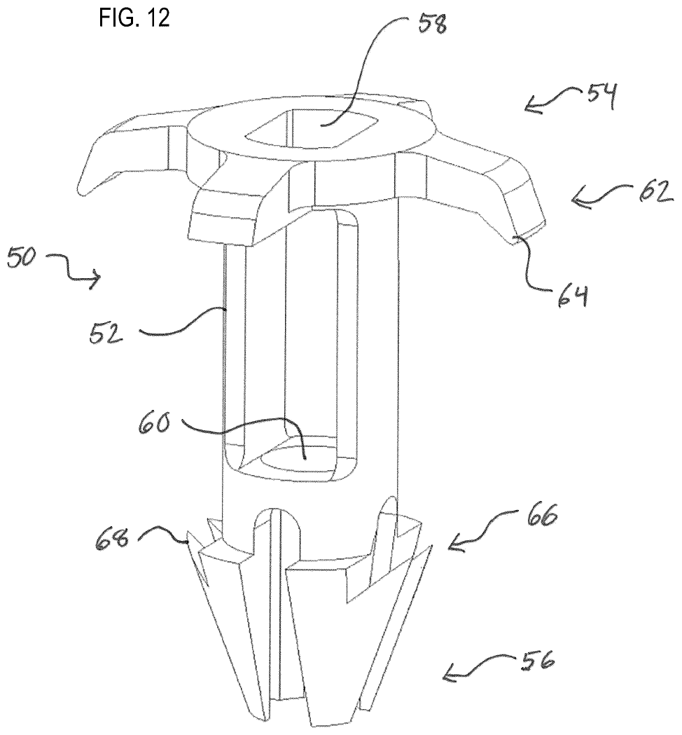

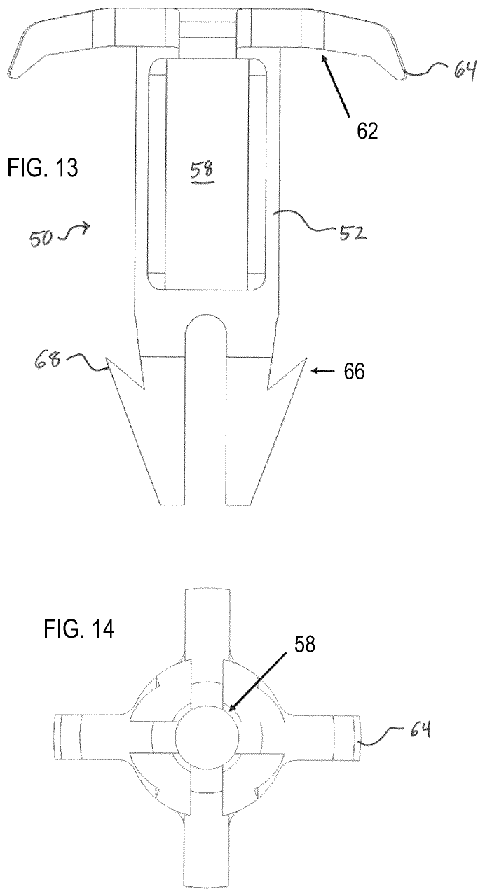

Referring to FIGS. 12 to 14, there is illustrated a graft fixation device 50, such as for medial graft fixation. Hence, in some embodiments, graft fixation device 50 can be referred to as a medial graft fixation device. In some embodiments, graft fixation device 50 can be referred to as an anchor. The fixation device 50 can include a body or joining feature 52 extending between a proximal end 54 and a distal end 56. A central cannulation or aperture or channel 58 extends axially through the main body or joining mechanism 52 to an interior distal surface 60. Central cannulation 58 is configured to receive a driver tool (not illustrated) that can impart distal force against the distal surface 60 to drive the fixation device 50 into a graft, soft tissue or bone depending upon the intended application.

A proximal gripping feature or mechanism 62 is provided to secure the graft. Gripping feature 62 may include a plurality of radially outwardly extending arms 64 such as at least two or four or more arms 64. Distal end 56 may be provided with a distal gripping feature or mechanism 66 such as a plurality of barbs 68. Medial graft fixation device 50 may comprise any of the variety of materials, such as titanium, stainless steel or resorbable polymers known in the art, depending upon the desired performance.

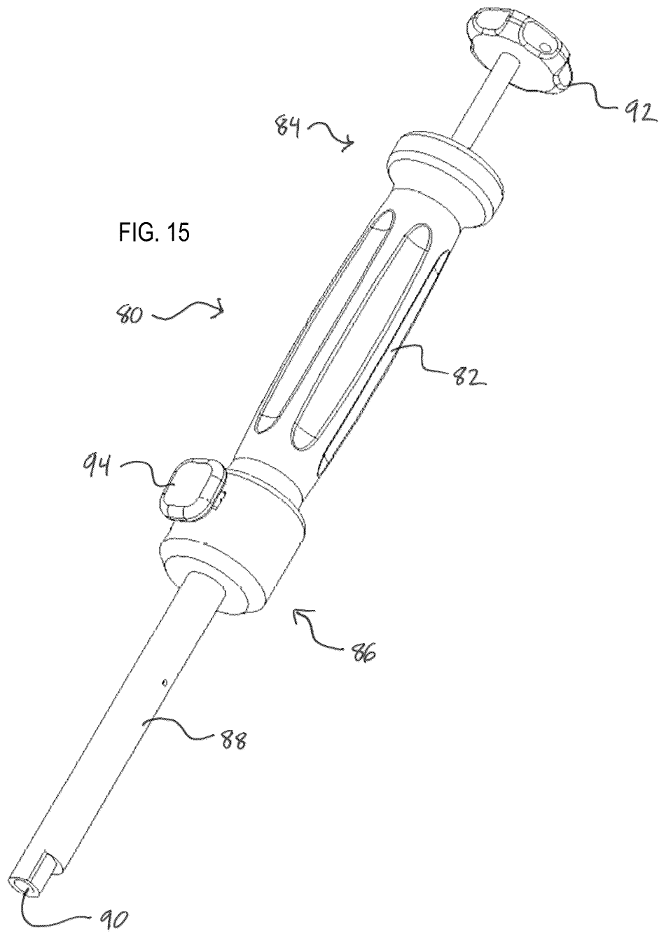

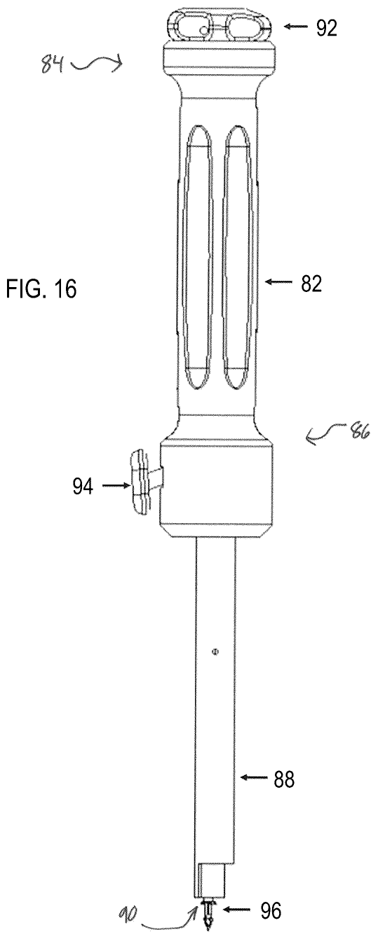

Referring to FIGS. 15 and 16, there is illustrated an anchor deployment device, such as a medial graft fixation deployment device 80. The deployment device 80 includes a handle 82 extending between a proximal end 84 and a handle distal end 86. An outer sheath 88 extends from the handle distal end 86 and terminates in a tack deployment aperture 90. In some embodiments, aperture 90 can be referred to as an anchor deployment aperture. A deployment control or insertion actuating feature or mechanism 92 may be actuated to deploy one or more tacks or anchors 96 through tack deployment aperture 90. In some cases, a tack or anchor 96 can be referred to as a medial graft fixation device.

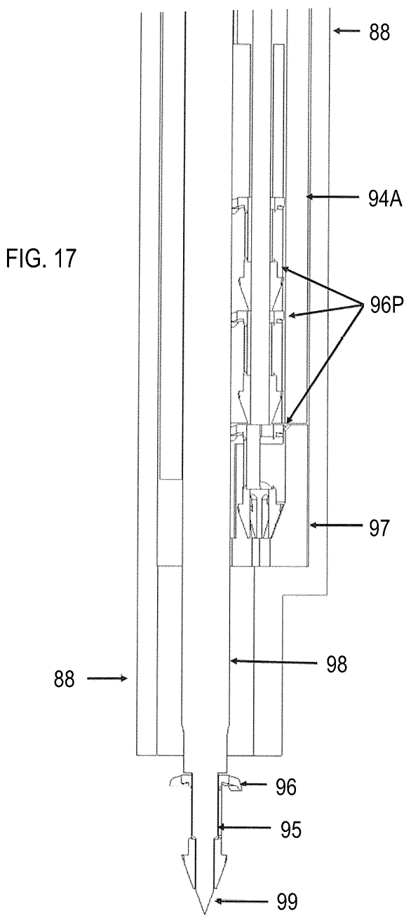

In some implementations of the invention, the graft deployment device 80 is configured to deploy at least two or three or four or more tacks or anchors for graft and or tissue attachment. A plurality of tacks or anchors may be stored within the handle and configured for sequential deployment through tack deployment aperture 90 by actuation of deployment control to deploy a first tack or anchor, followed by actuation of a reloading control 94 to advance a second tack or anchor into a ready position for deployment by the deployment control 92, without the need to remove the deployment device from the patient or reload the device between tack or anchor deployments. In some cases, reloading control 94 can be referred to as a reloading trigger feature or mechanism. In some cases, reloading control 94 can be in operative association with a cartridge (e.g. cartridge 94A depicted in FIG. 17).

Referring to FIG. 17, a plurality of pre-loaded graft fixation devices (e.g., tacks or anchors) 96P may be contained within the handle or outer sheath 88, such as within a cartridge 94 or within a tack or anchor lumen without a cartridge. In the illustrated embodiment, the pre-loaded fixation devices 96P are coaxially aligned and configured for sequential distal advance into the reloading feature or mechanism 97. Actuation of a reloading control (e.g., a button or trigger) advances a pre-loaded fixation device 96P into a deployment ready position such as within the insertion shaft 98 (e.g. as illustrated by fixation device 96) from which it may be distally advanced through the tack or anchor deployment aperture 90, such as by distal, axial advance of an awl 99. Following deployment of a given fixation device 96, the awl may be proximally retracted and the reloading feature or mechanism 97 actuated to advance a second pre-loaded fixation device 96P into a ready position within the cannulated insertion shaft 98 distally of the awl 99. The awl 99 can then be advanced distally to extend the fixation device into a deployment position (e.g. as illustrated by fixation device 96). In some cases, a fixation device 96 can include an engagement feature or mechanism 95, which can operate to engage with the awl 99.

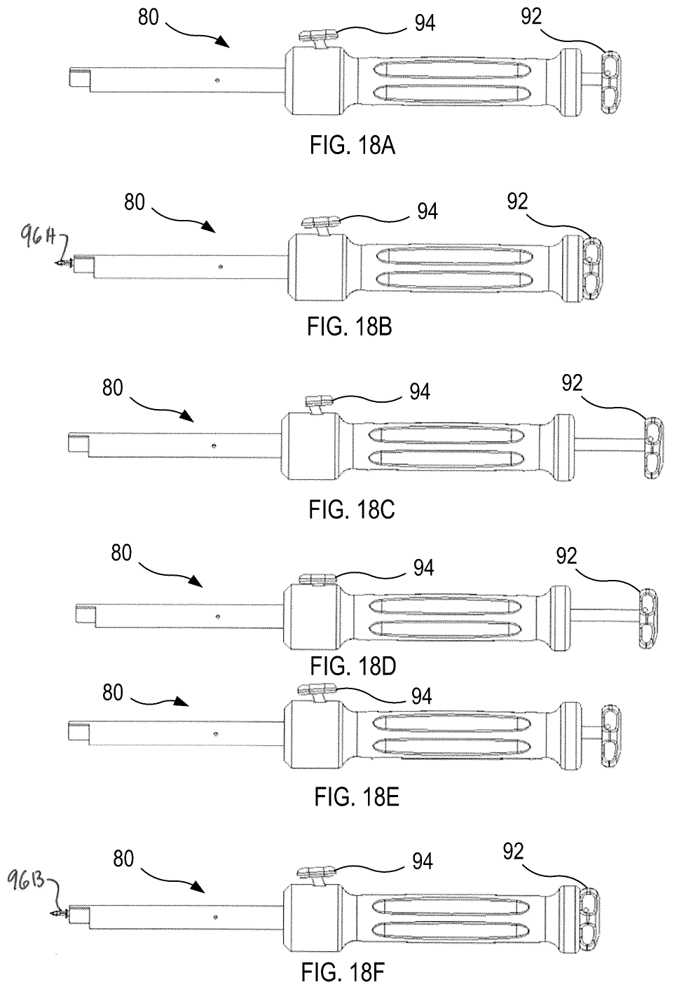

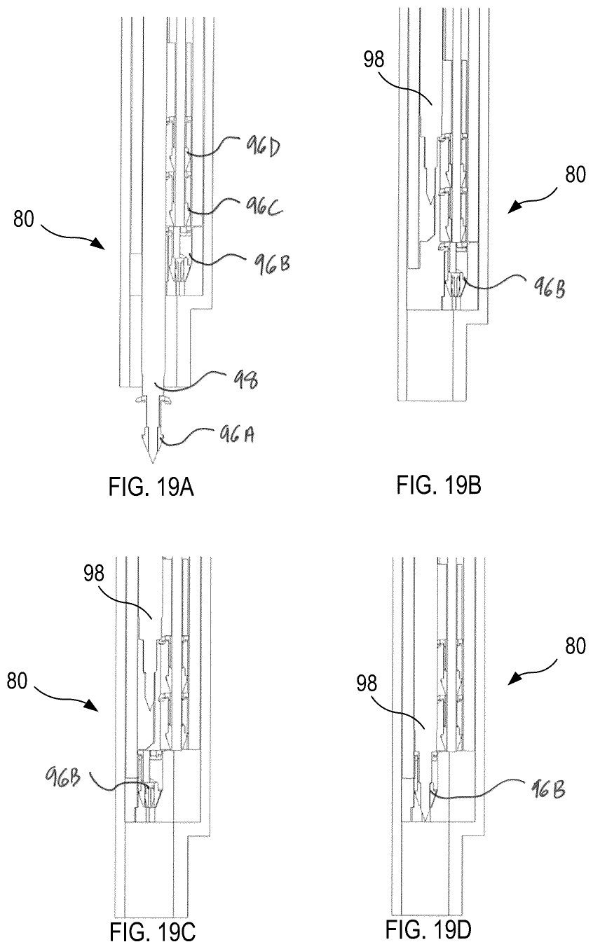

FIGS. 18A to 19D show a sequence series of views of the deployment device 80, deploying more than one fixation device or anchor. FIG. 18A depicts deployment device 80 in an initial configuration. As shown in FIG. 18B, an insertion actuating feature or mechanism 92 can be used to advance an insertion shaft, thus driving a fixation device 96A into a desired position. As shown in FIG. 18C, the actuating mechanism 92 can be retracted, thus withdrawing the insertion shaft to a reset position (e.g. after initial anchor 96A has been placed in the patient). As shown in FIG. 18D, the reloading trigger feature or mechanism 94 can be actuated, as the insertion shaft is returned to the initial position. As shown in FIG. 18E, the insertion actuating mechanism 92 can be advanced distally. As shown in FIG. 18F, this process can be repeated until all anchors (e.g. next anchor 96B and any additional anchors) are placed as desired. Relatedly, FIG. 19A shows a first fixation device 96A that has been deployed by the awl 98, and additional fixation devices 96 B, C and D stored within the deployment device 80 for subsequent deployment. In this way, anchor 96A can be inserted. As shown in FIG. 19B, the insertion shaft can be withdrawn or retracted. As shown in FIG. 19C, the next fixation device 96B can be placed in the path of the shaft 98 (e.g. by actuation of a reloading feature or mechanism). As shown in FIG. 19D, the shaft 98 can be advanced, engaging with the fixation device 96B and returning to the initial position.

Although the fixation devices 96 in the illustrated implementation are stored in a coaxial, linear configuration, other configurations may alternatively be used. For example a plurality of fixation devices 96 may by spaced apart circumferentially in a rotatable support like a plurality of chambers in a revolver cylinder.

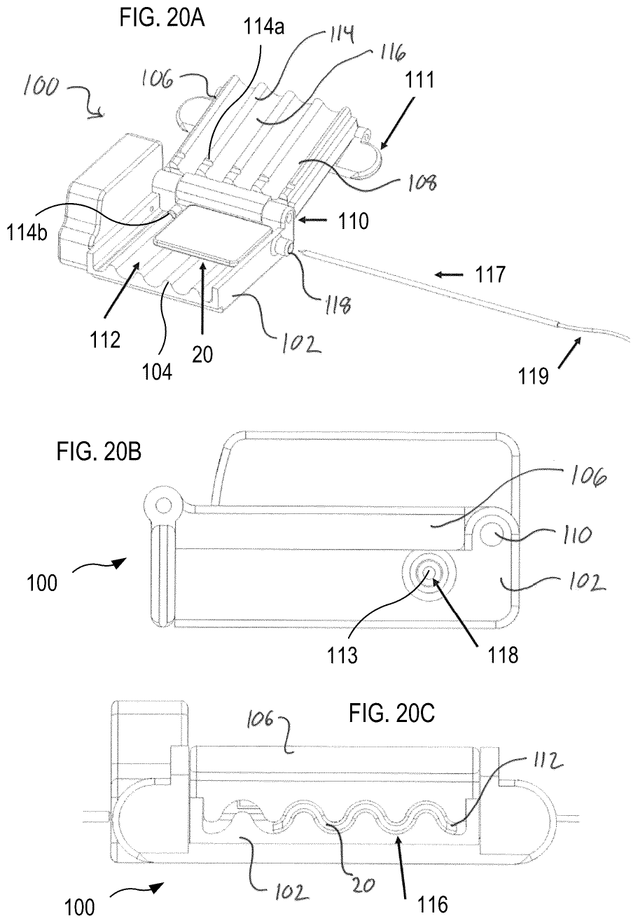

Referring to FIGS. 20A to 20C, there is illustrated a suture loading block 100 configured to facilitate stitching a suture in a sinusoidal pattern through a graft. In some cases, suture loading block 100 can be referred to as a suture loading block assembly or a suture loading block device. The suture loading block 100 includes a base or bottom body 102 having a first contoured surface 104. The block 100 also includes a lid or top body 106 having a second complementary contoured surface 108 (e.g. complementary to the shape of the first contoured surface 104) and that is rotatable about a hinge feature or mechanism 110. The first and second complementary contoured surfaces (104, 108) may be brought into proximity with each other, spaced apart by a gap sufficient to accommodate a graft 20 within a graft space 112. Each of the contoured surfaces 104 and 108 comprise a plurality of complementary axially extending ridges 114 separated by axially extending valleys 116.

An aperture or cannula 118 of the base 102 lies on an axis extending at a perpendicular to the longitudinal axis of the ridges 114 and valleys 116, configured to receive a needle 117 and associated suture 119 along a path through the corrugated graft 20. The ridges of the block 100 can include first ridge troughs 114a and second ridge troughs 114b configured to receive the needle and suture when the block 100 is in a closed state. For example, the first ridge troughs 114a and the second ridge troughs 114b can provide a passageway 113 in line with the cannula 118 through or in which the needle and/or suture may be passed or positioned. The needle and suture can be passed through the troughs along the path and the suture can thereby be loaded onto the graft. The lid 106 may thereafter be opened (e.g. by unlocking a locking mechanism 111 which can be locked when the block is in a closed state), to release the graft 20 having the suture extending through the graft in a sinusoidal pattern.

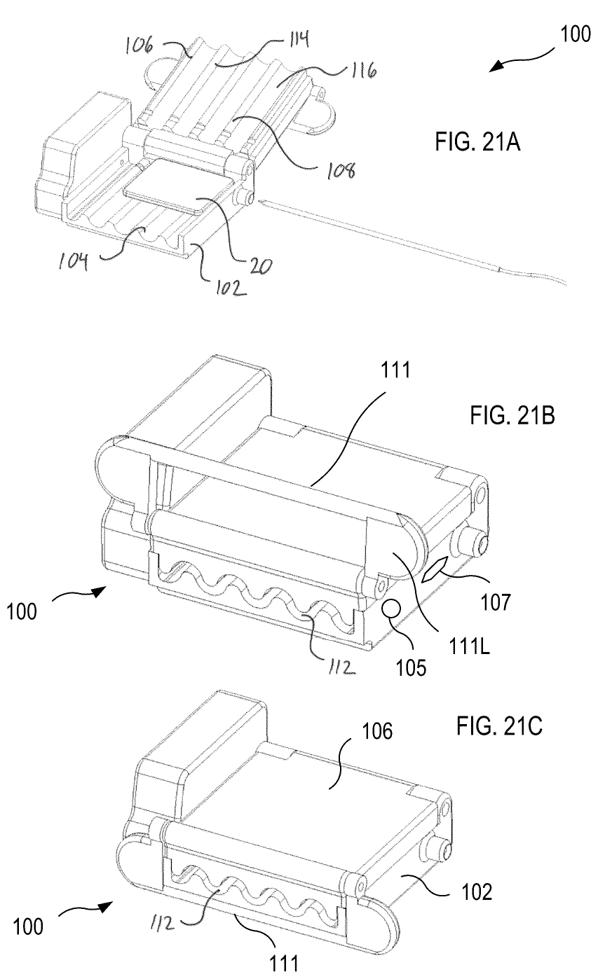

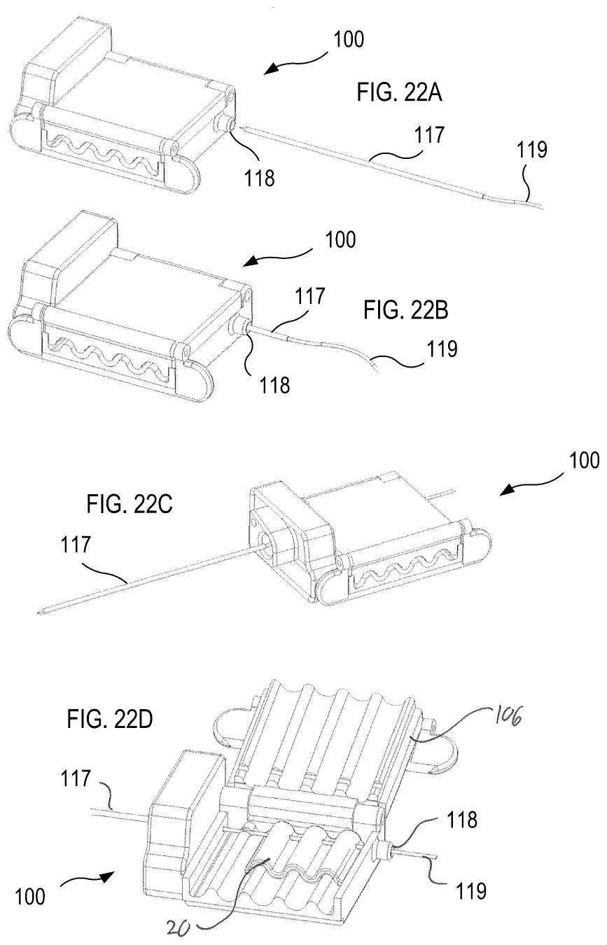

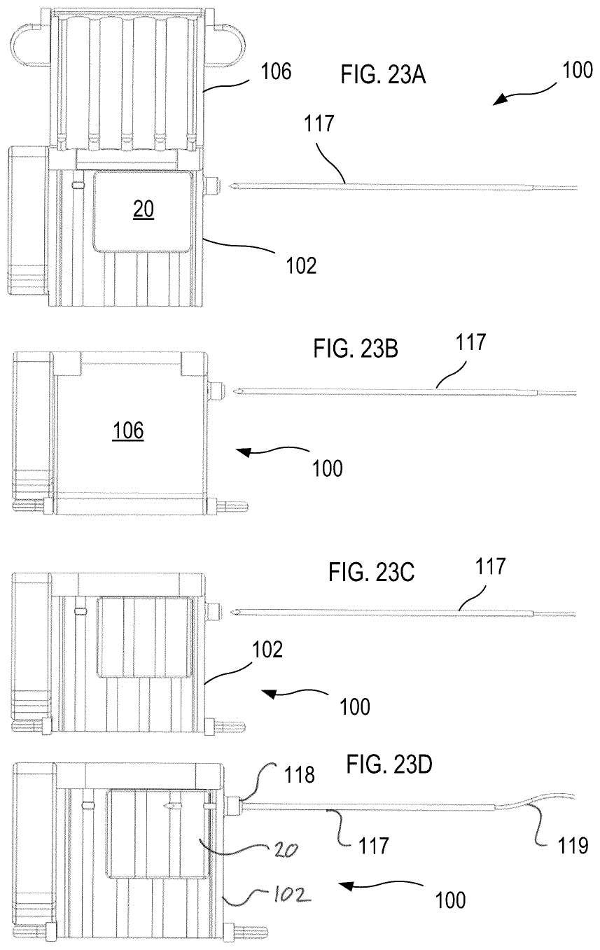

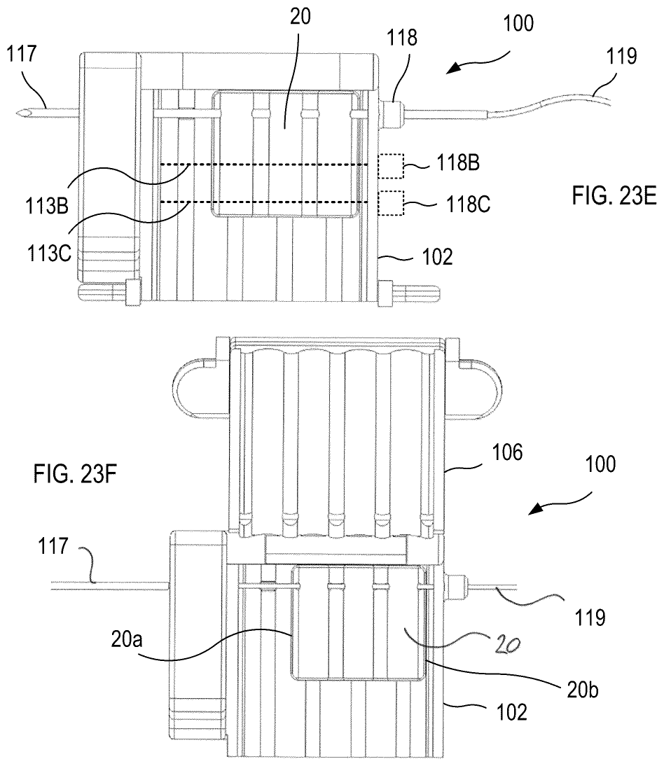

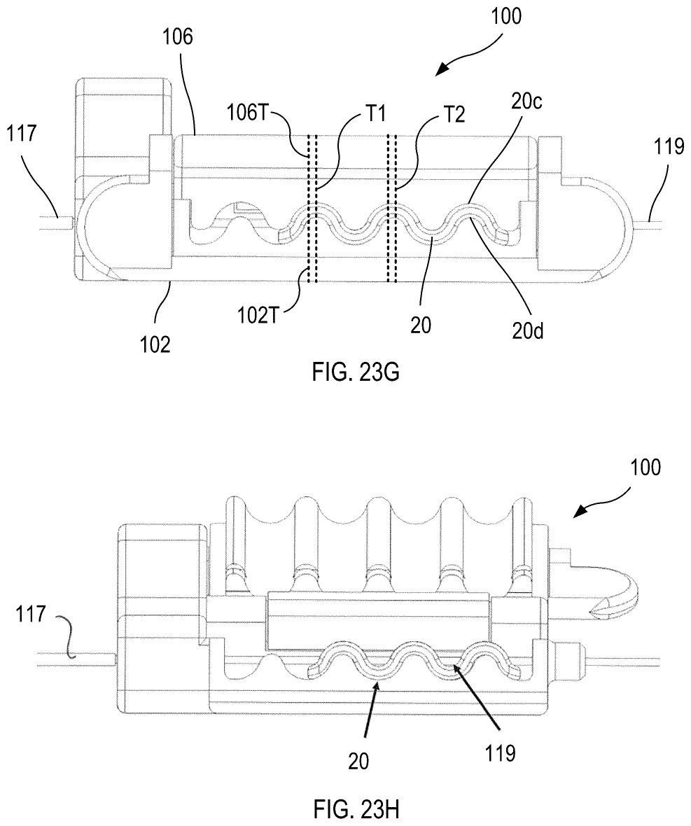

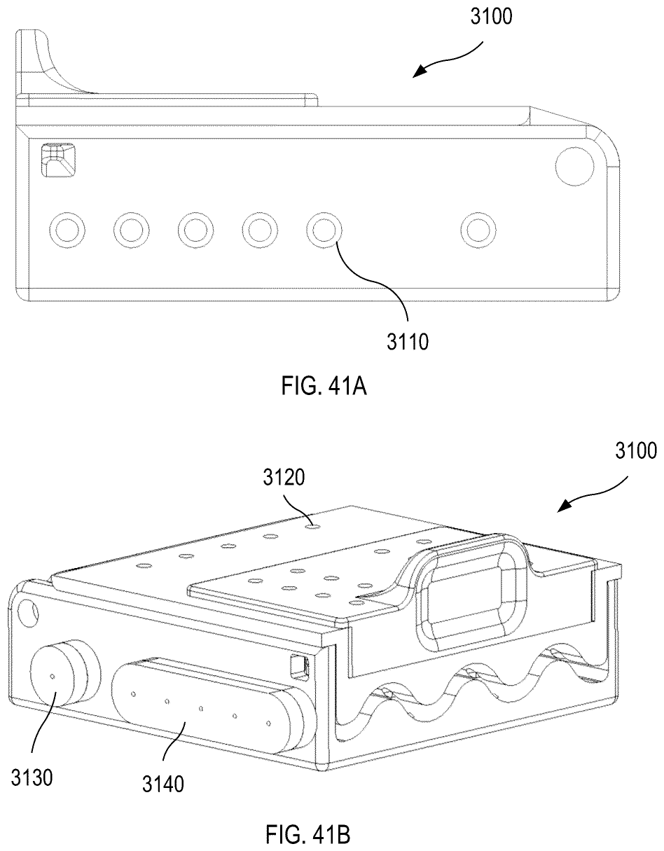

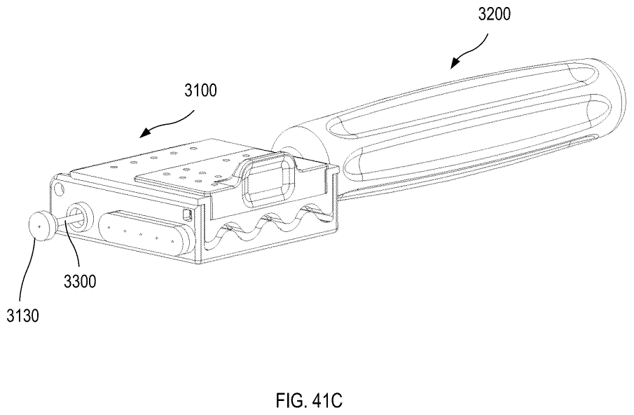

FIGS. 21A to 23H show different stages and views in the operation of the suture loading block 100. As illustrated in FIG. 21A, the graft 20 can be placed on the base 102 (e.g. so that it can be placed into the into the graft space). As shown in FIG. 21B, the lid 106 can be closed, causing the graft shaping features (e.g. ridges and valleys) to shape the graft (e.g. within the graft space 112). In FIG. 21B, the locking mechanism 111 is in an unlocked configuration. The locking mechanism 111 may include a lever 111L. In some cases, a locking mechanism 111 can be engaged automatically with a spring load when the loading block is closed and can be released with a button. In some cases, the block 100 can be spring loaded to open when the locking mechanism 111 is released. Accordingly, as shown here, block 100 includes a spring mechanism 105 and a button mechanism 107. As shown in FIG. 21C, the locking mechanism can be engaged, so as to lock the lid and the base together. As shown in FIGS. 22A and 22B, the needle 117 can be loaded with the desired suture 119 and can be passed through the cannula 118 through the block and the graft. As shown in FIG. 22C, the needle 117 can be passed through and removed from the far side of the loading block 100. As shown in FIG. 22D, the locking feature or mechanism 111 can be disengaged, and thereafter the loaded graft (e.g. graft 20 loaded with the suture 119) can be removed from the block 100. FIGS. 23A to 23H depict a process whereby the block 100 is used in loading the graft 20 with the suture 119. The lid 106 is removed from view in FIGS. 23C to 23E to help illustrate details of the graft loading method, whereby the suture 119 can be passed through the graft 20 in a sinusoidal pattern. As depicted in FIG. 23E, the contoured surfaces of the lid and base can include any number of additional troughs defining any number of additional respective passageways 113B, 113C through which additional needles loaded with additional sutures may be passed when the loading block is in a closed configuration. For example, the first contoured surface can include a plurality of third ridge trough and the second contoured surface can include a plurality of fourth ridge troughs, such that the plurality of third ridge troughs and the plurality of fourth ridge troughs provide a second passageway 113B that is configured to receive a second needle loaded with a second suture therethrough when the loading block is in the closed configuration. Likewise, the first contoured surface can include a plurality of fifth ridge trough and the second contoured surface can include a plurality of sixth ridge troughs, such that the plurality of fifth ridge troughs and the plurality of sixth ridge troughs provide a third passageway 113C that is configured to receive a third needle loaded with a third suture therethrough when the loading block is in the closed configuration. Relatedly, the base 102 can include corresponding cannulas 118B, 118C aligned with the passageways 113B, 1103 respectively. As shown in FIG. 23F, the cannulas and passageways are aligned in a perpendicular orientation to the ridges and valleys of the contoured surfaces, so that when the needles and sutures are passed therethrough, the sutures are also aligned in a perpendicular orientation relative to the side edges (e.g. 20a, 20b) of the graft. In some cases, as shown in FIG. 23G, the lid 106 and base 102 can be provided with transverse apertures or channels 106T, 102T, so as to provide traverse passageways (e.g. T1, T2) through which needle and/or sutures may be passed. As depicted here, the traverse passageways can be perpendicular to the top and bottom surfaces (e.g. 20c, 20d) of the graft 20. Analogous apertures or holes are depicted in FIG. 41B (e.g. top holes 3120).

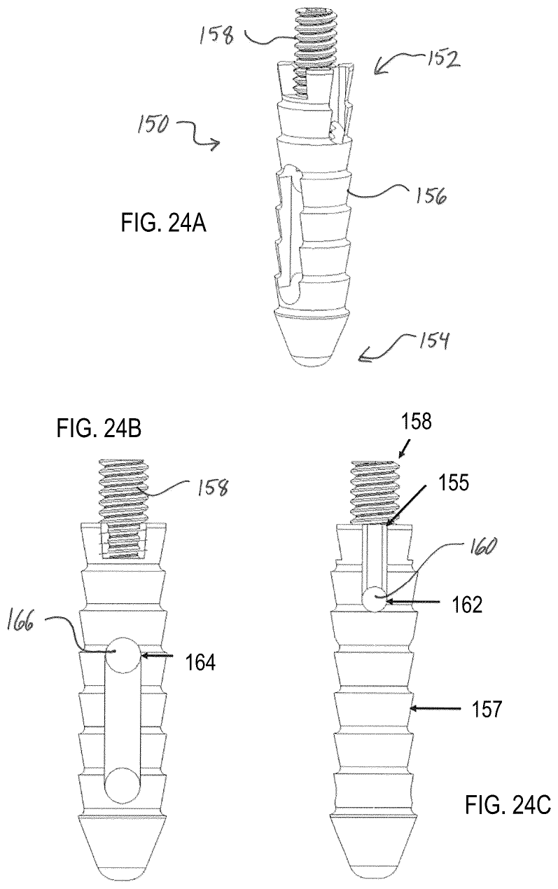

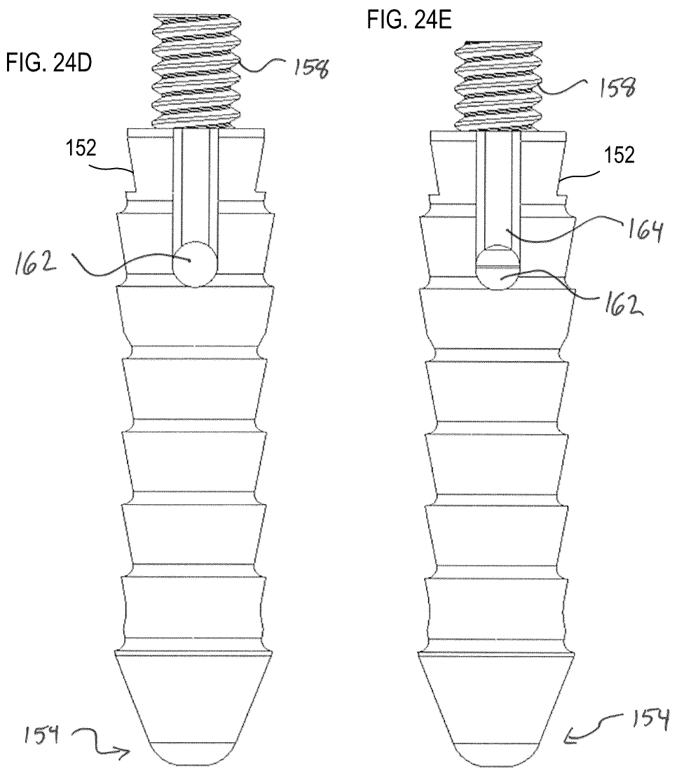

Referring to FIGS. 24A to 24E, there is illustrated an anchor such as a lateral anchor 150 configured for graft suture fixation. The lateral anchor 150 may be configured to be rotated or hammered into bone, and may be provided with a plurality of retention surface structures or exterior fixation features or mechanisms 157 such as annular ridges or a helical thread. The anchor 150 may comprise any of a variety of pawn anchor materials including metals such as titanium or scale of steel, or polymers such as polyether ether ketone (PEEK). In some cases, anchor 150 can be referred to as a suture anchor.

The anchor 150 comprises a body 156 extending between a proximal end 152 and a distal end 154. Anchor body 156 is provided with a suture fixation feature or mechanism 158 which may be in the form of a threaded shaft or screw. The suture fixation feature or mechanism 158 can be configured to compress against and lock a graft suture 160 passing through a graft suture lumen or passing feature or hole 162, which may extend transversely through the body 156. In some cases, the suture fixation feature or mechanism 158 can engage with the body 158 via an inserter connection feature or mechanism 155.

A repair suture passing feature 164 such as a transverse lumen 166 may extend through the body 156 such as transverse to a longitudinal axis of the body 156 and also transfers to the axis of the graft suture lumen 162. As shown in FIGS. 24D and 24E, the screw or suture fixation mechanism 158 can be advanced into the proximal end 152 of the body so as to lock the graft suture.

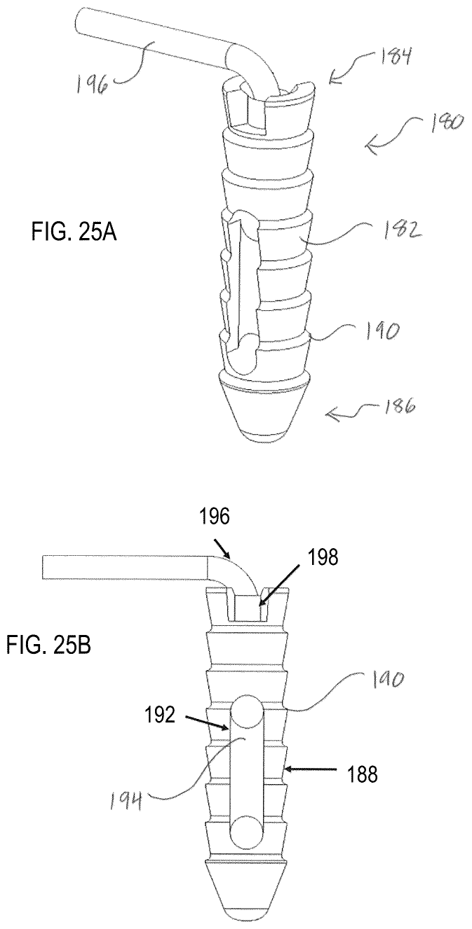

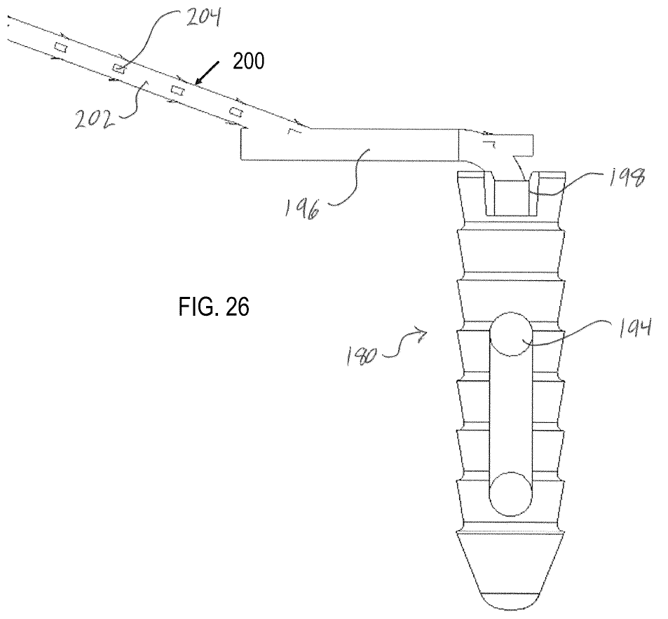

Referring to FIGS. 25A and 25B, there is illustrated an anchor such as a lateral anchor having a feature for attachment to a barbed suture or hook (e.g. the barbed suture shown in FIG. 26). The anchor 180 includes a body 182 extending between a proximal end 184 and a distal end 186. Body 182 may comprise any of a variety of materials depending upon the intended use. If intended for implantation into bone, a relatively rigid material such as stainless steel, titanium, or polyether ether ketone (PEEK) may be used.

Exterior fixation feature or mechanism 188 such as a plurality of ramped surfaces, annular flanges or helical threads 190 may be provided for resisting proximal pullout of the installed anchor. A repair suture passing feature or mechanism 192 may be provided, for attaching the repair suture. The passing feature or mechanism 192 may comprise an aperture 194 for receiving the repair suture. A graft suture fixation feature or mechanism 196 may be provided for securing a graft suture. The graft suture fixation feature or mechanism 196 may be secured to the body 182 by inserter connection feature or mechanism 198 which may include glue, an eye, compression fit or other attachment feature or mechanism.

The graft suture fixation feature or mechanism 196 may comprise an elongate flexible structure capable of entangling or engaging a barbed suture or suture with at least one or more hooks or engagement structures or wire mesh or woven or nonwoven fabric that allow the suture to advance in a distal direction along the outside or through the fixation feature or mechanism 196 and resist proximal removal of the suture from the fixation feature or mechanism 196.

Referring to FIG. 26, the barbed suture 200 is illustrated as engaged with the suture fixation feature or mechanism 196. Barbed suture 200 includes an elongate flexible filament body 202 having a plurality of barbs 204 which incline radially outwardly in the proximal direction to resist proximal detachment of the suture 200 from the attachment feature or mechanism 196.

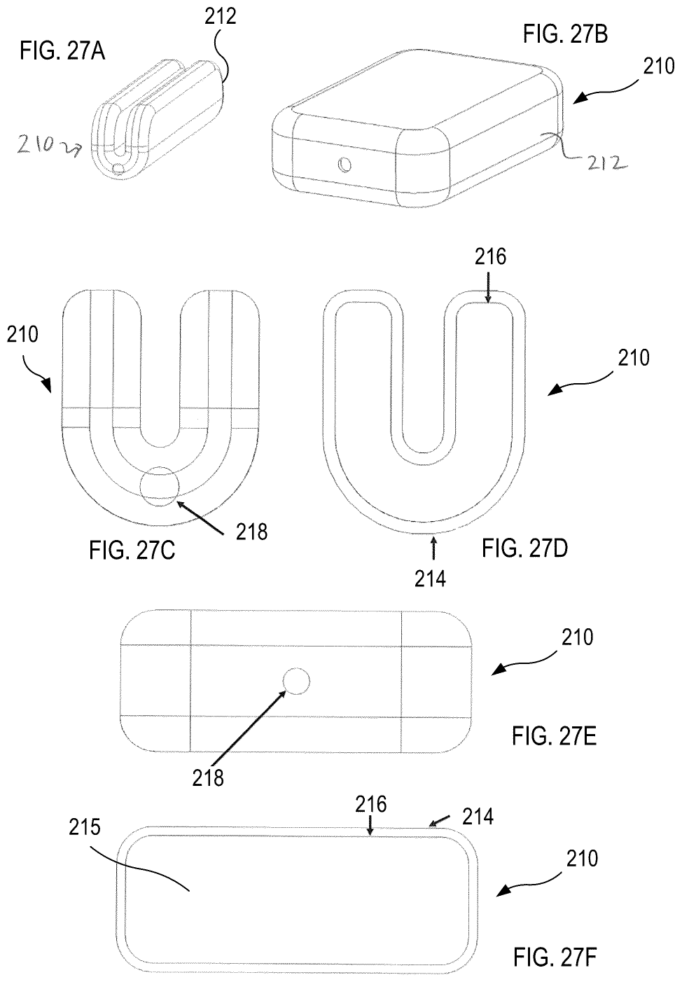

Referring to FIGS. 27A to 27F, there is illustrated a spacer 210 which may be implanted in between adjacent anatomical structures and enlarged to increase or control the space between the anatomical structures. In some implementations, the spacer 210 may be a joint spacer for treatment of a joint such as a subacromial spacer, a glenohumeral spacer, or a spacer for another joint such as a knee, hip, ankle, or hand joint.

The body 212 of the spacer 210 may comprise an exterior layer 214 inclosing and interior volume 215 containing a bulking media 216, as depicted in FIG. 27D. In some embodiments, both the exterior layer 214 and the bulking media 216 will dissolve over time. Suitable exterior layers 214 may comprise polylactic acid (PLA), polyglycolic acid (PGA,) or another bioresorbable polymer. Suitable bulking media may comprise any of a variety of gels such as hydrogels or bioactive glass for example. In some embodiments, a desired resorption time can have a range of 3 to 18 months. In some embodiments, a desired resorption time can have a range of 6 to 9 months. The body 212 may have two opposing generally planer top and bottom walls attached by a peripheral boundary to produce a flat pillow shape. In some embodiments, one or more walls or sides can have a planned curvature (e.g. a simple arc conforming to anatomy). In some embodiments, the body 212 is approximately rectangular having dimensions of no greater than about 6″ by 6″, and in some implementations no greater than about 3″ by 5″ or 2″ by 3″. In some embodiments, the expanded state thickness is generally within the range of from about 0.5 cm to about 2.5 cm, and in some implementations between about 4 mm and about 10 mm.

In some embodiments, the body 212 is foldable or rollable between a first, low profile configuration to facilitate implantation, illustrated in FIG. 27A (e.g. unexpanded) and a second deployed configuration as illustrated in FIGS. 27B (e.g. expanded).

In one implementation, the media is a solid hydrogel which will swell to a known volume upon exposure to water or other fluid. The spacer 210 may be positioned between adjacent anatomical structures, opened up to the deployed (e.g., flat) orientation followed by the introduction of a fluid such as saline through an inlet 218 as shown in FIG. 27C to cause the bulking media to swell (and e.g. causing the body to expand). In some embodiments, the inlet 218 may not need a valve to resist escape of the bulking media, which transitions from dry solid to a solid gel following rehydration.

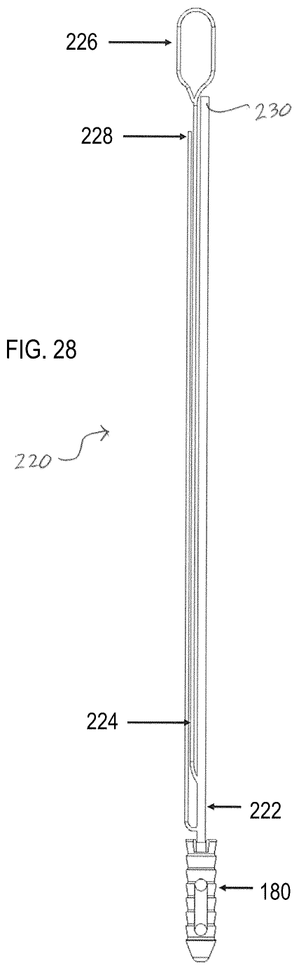

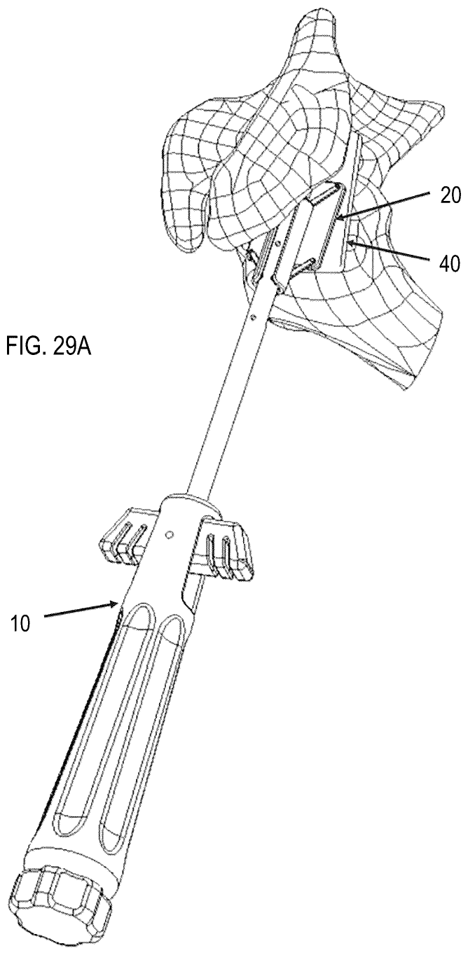

Referring to FIG. 28 there is illustrated a loading assembly 220 which may be used for loading a lateral anchor with a barbed graft fixation suture. In some embodiments, loading assembly 220 can be referred to as an anchor loading assembly. An anchor 180 is secured to the distal end of an elongate flexible braid 222. A passing suture 224 extends between the proximal end 228 of the assembly and a distal passing loop 226. The passing suture 224 loops through the braid 222 adjacent to the anchor 180. As illustrated, both the proximal end 228 and the distal passing loop 226 initially extend proximally from the anchor. Proximal retraction or pulling of the proximal end 228 following implantation of the anchor 180 draws the passing loop 226 in the distal direction towards the anchor 180. The braid 222 may extend proximally from the anchor to a proximal end 230 to allow the physician to grasp it and place proximal tension on the anchor 180. After a suture snared by the passing loop 226 has been drawn distally towards the anchor 180, the proximal tail of the braid 222 may be severed and removed. In this way, a loading method for a lateral anchor with a barbed graft suture fixation can be accomplished.

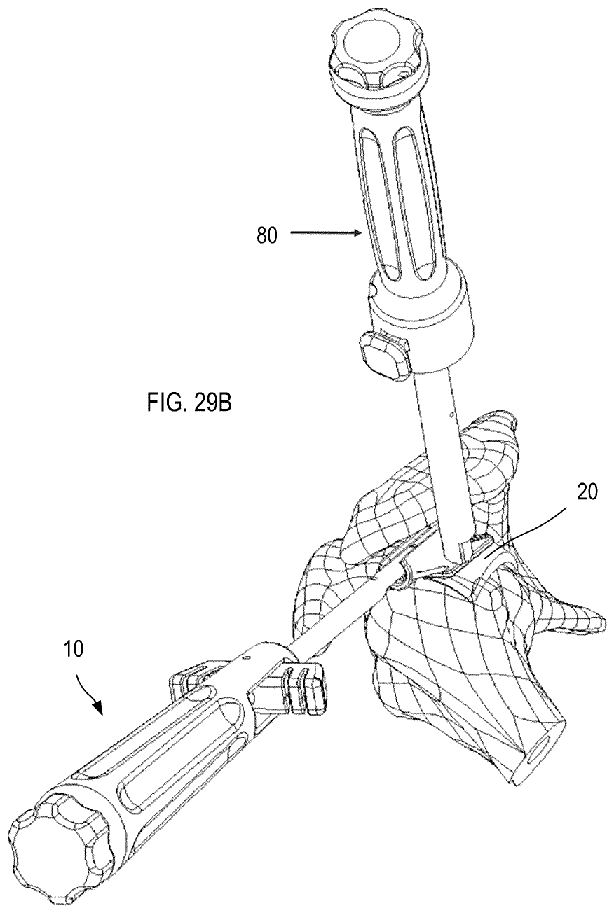

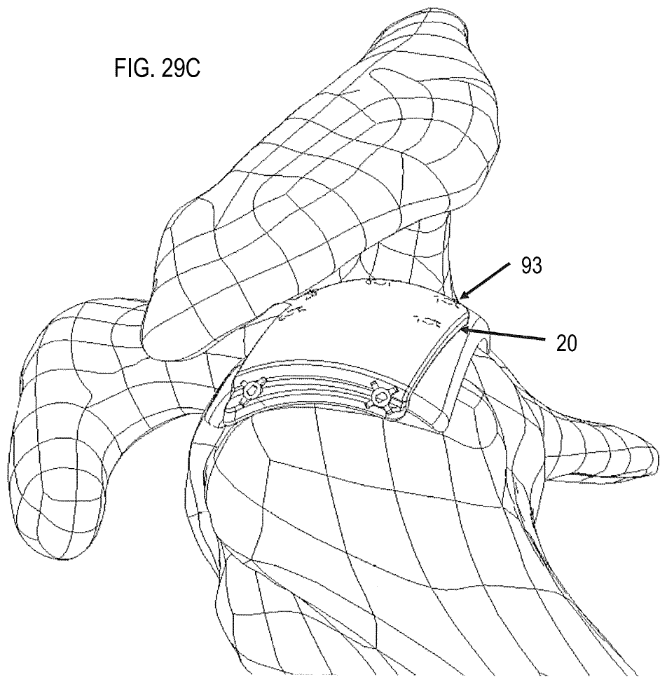

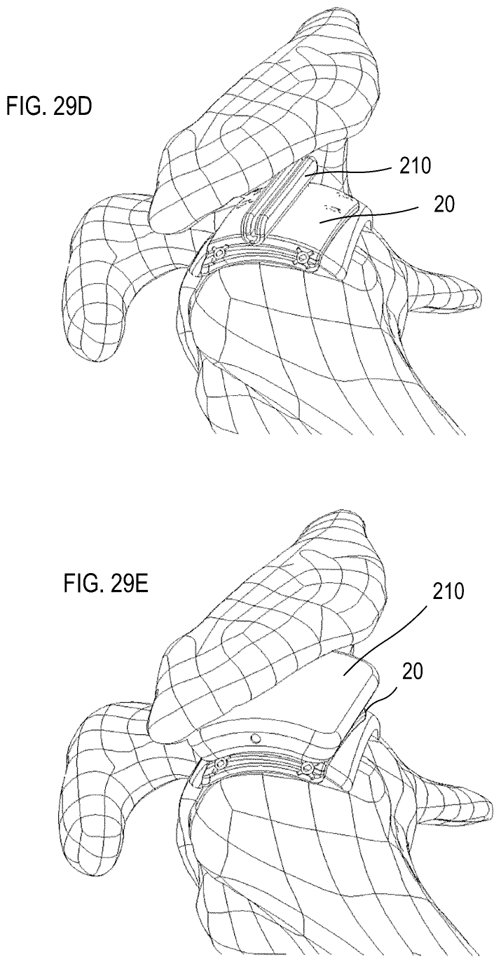

FIGS. 29A to 29E illustrate relative to the anatomy the various steps of inserting the graft, graft fixation, and inserting and hydrating the spacer. As shown in FIG. 29A, a deployment device 10 can be used to deliver a graft 20 to a surgical treatment location (e.g. rotator cuff 40) of a patient. As shown in FIG. 29B, a fixation deployment device 80 can be used to deliver fixation devices to secure the graft 20 with the patient (e.g. while the graft is held in place using the deployment device 10). As shown in FIG. 29C, fixation devices 93 such as anchors can secure the graft 20 with the patient tissue (e.g. bone). As shown in FIG. 29D, a spacer 210 (e.g. in unexpanded configuration) can be inserted or placed adjacent to the graft 20. As shown in FIG. 29E, the spacer 210 can be converted to an expanded configuration (e.g. by hydrating the spacer).

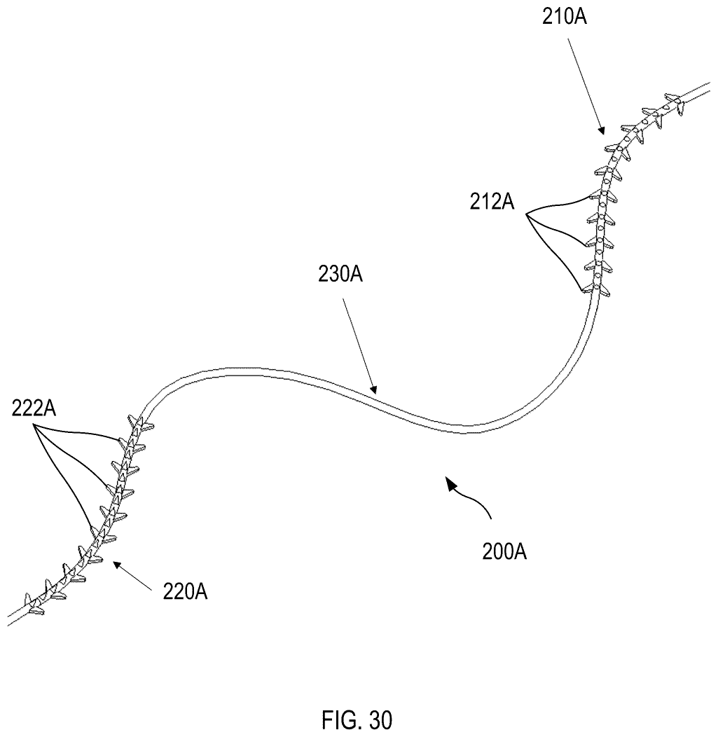

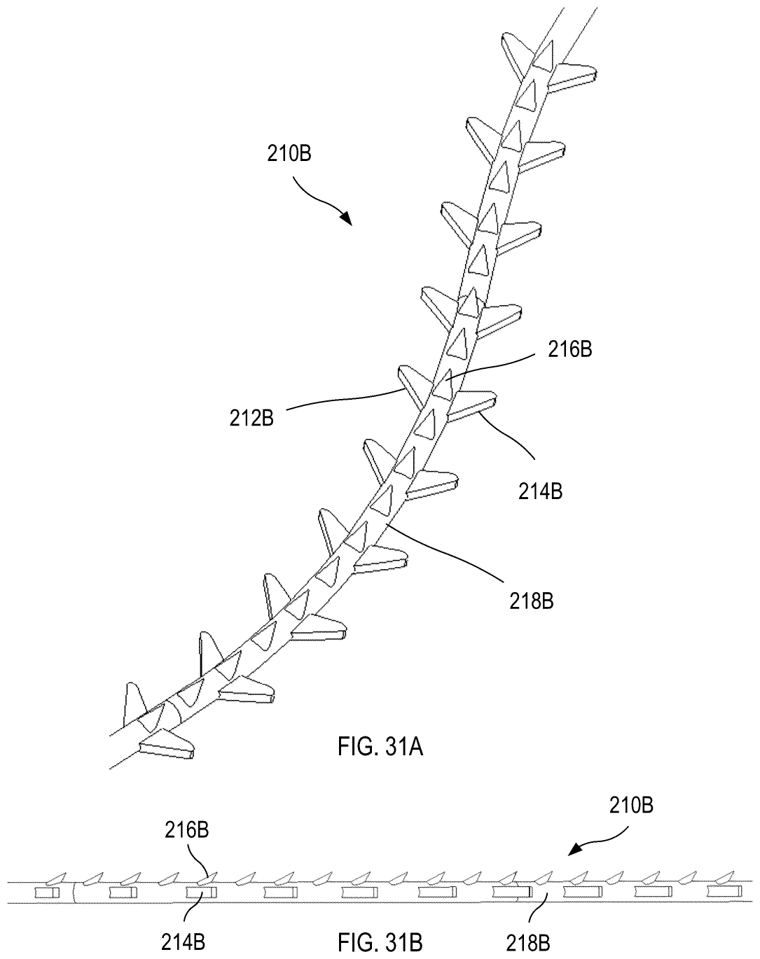

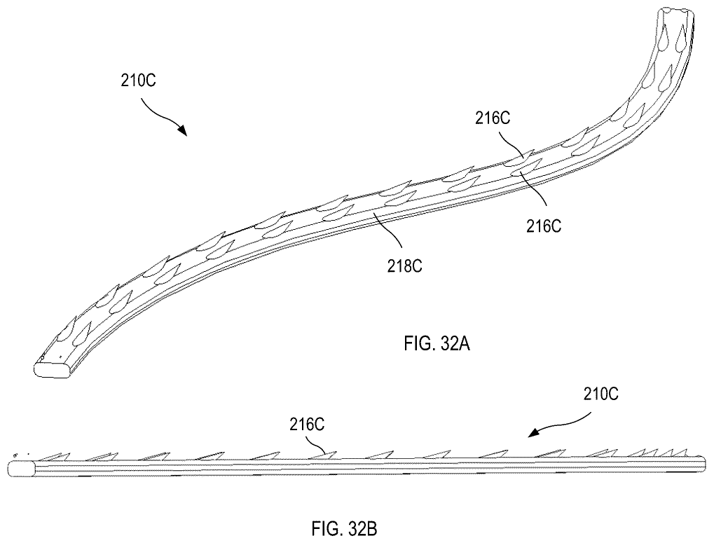

FIGS. 30 to 32B depict aspects of barbed suture devices which can be added to or used in conjunction with a system. Such barbed suture devices can be used in any of the places of the procedures discussed elsewhere herein which involve sutures or barbed sutures (e.g. suture loading block or one of the graft suture fixation devices) or independently in the system to assist in graft fixation. For example, any of the barbed sutures or aspects thereof shown in FIGS. 30 to 32B can be used with the graft suture fixation mechanism illustrated in FIG. 26. As shown in FIG. 30, a barbed suture device 200A can have a first barbed section 210A with individual barbs 212A arranged in a first laterally pointing configuration, and a second barbed section 220A with individual barbs 222A arranged in a second laterally pointing configuration that is opposite directionally from that of the first laterally pointing configuration. The barbed suture device 200A can also include a smooth section 230A disposed between the first barbed section 210A and the second barbed section 220A, that is centrally located on the device and devoid of any barbs. Hence, exemplary barbed suture device designs can be characterized by having barbed sections with opposite directionality joined by a smooth section of suture. FIGS. 31A and 31B depict aspects of a barbed section 210B of a barbed suture. As shown here, barbed section 210B can include a series of opposing lateral barbs or fins 212B, 214B and a series of dorsal barbs or fins 216B disposed along a suture body 218B. As shown here, the dorsal barbs or fins 216 can be arranged in an orientation that is perpendicular to that of the lateral barbs or fins 212B, 214B. FIGS. 32A and 32B depict aspects of a barbed section 210C of a barbed suture. As shown here, barbed section 210C can include a series of repeating dual dorsal barbs or fins 216C disposed along a flattened tape suture body 218C.

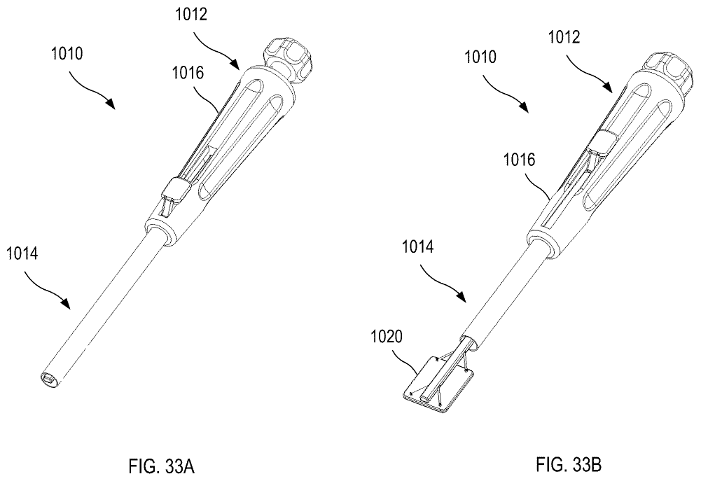

FIGS. 33A and 33B depict aspects of a graft deployment device 1010 having a proximal end 1012 and a distal end 1014. A handle 1016 of the device supports a graft deployment control 1018, which may be a lever, rotatable knob, slider, switch, or other control. The distal end 1014 carries a graft 1020 which can be in a low profile (e.g. initial) configuration for advancement to the treatment site, and as shown in FIG. 2 can be in an expanded configuration for implantation and attachment of the graft at the treatment site.

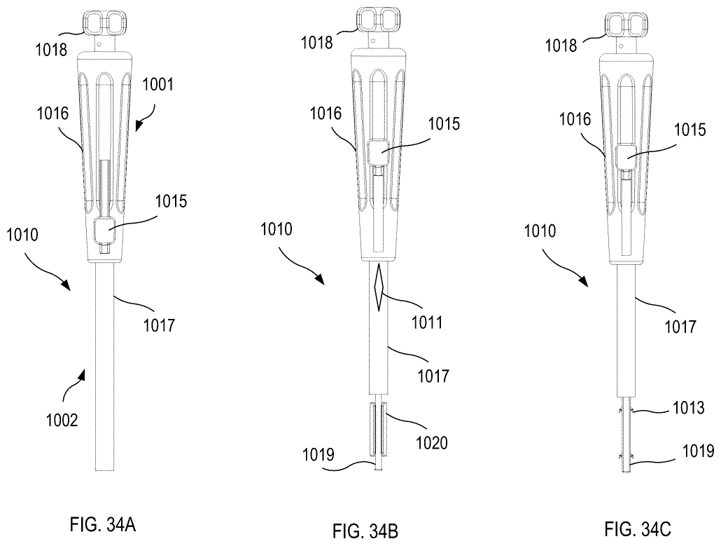

FIGS. 34A to 34C depict aspects of a graft deployment device 1010. FIG. 34A illustrates the graft deployment device 1010 in an initial configuration, having a graft deployment control or knob or trigger 1018, a handle 1016, a sheath lever 1015, and a sheath 1017. In some cases, handle 1016 of the device supports a graft deployment control 1018, which may be a lever, rotatable knob, slider, switch, trigger, or other control. In some embodiments, the handle and graft deployment control can comprise a body of the device. The body 1001 of the device 1010 can be in operative association with an insertion mechanism 1002 of the device. The insertion mechanism 1002 can include the outer sheath 1017, a support shaft 1019 disposed at least partially within the outer sheath, and an expansion mechanism (e.g. wires 1013) coupled with the support shaft. The expansion mechanism can be configured to support and/or expand a graft 1020. In some cases, the device body 1001 includes a sheath lever 1015 configured to retract the outer sheath. FIG. 34B illustrates the graft deployment device in a second configuration, where the sheath 1017 has been retracted proximally (e.g. by actuation of the sheath lever 1015) and the shaft 1019 is exposed, with the graft 1020 at least partially wrapped about or otherwise loaded on the shaft 1019. In some embodiments, the outer sheath 1017 can be spring loaded (e.g. with a spring 1011). As depicted in FIG. 34C, the device 1010 can also include one or more wires 1013 by which the shaft 1019 can be coupled with the graft (not shown).

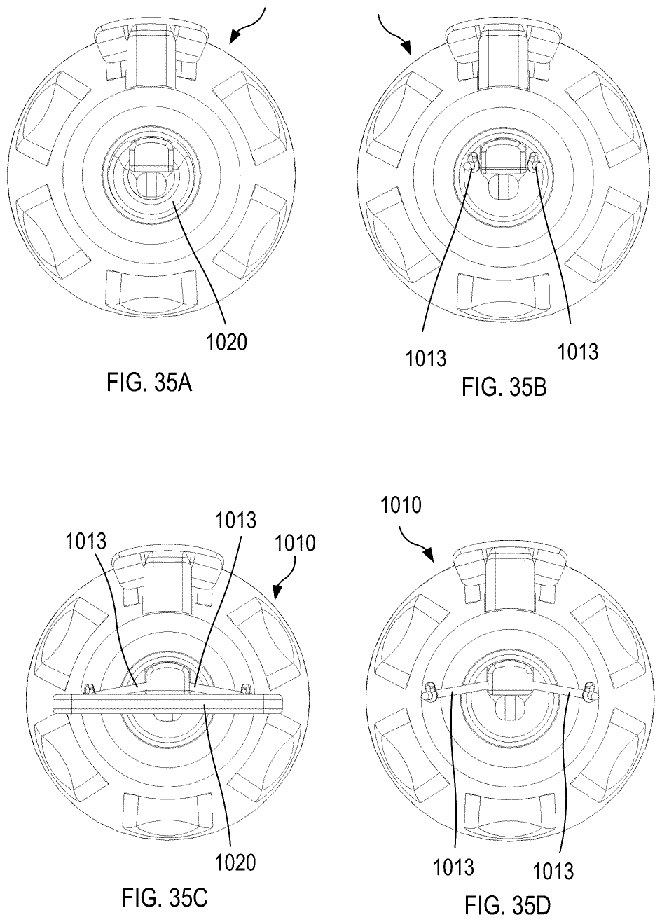

FIGS. 35A to 35D show an end view of a distal portion of the graft deployment device 1010, throughout various stages of a graft deployment process. In FIG. 35A, the wires are retracted and the graft 1020 is loaded on the device. FIG. 35B provides another view where the wires 1013 are retracted and the graft (not shown) is loaded on the device. In FIG. 35C, the wires 1013 support the graft 1020 which is now expanded. In FIG. 35D provides another view where the wires 1013 support the expanded graft (not shown).

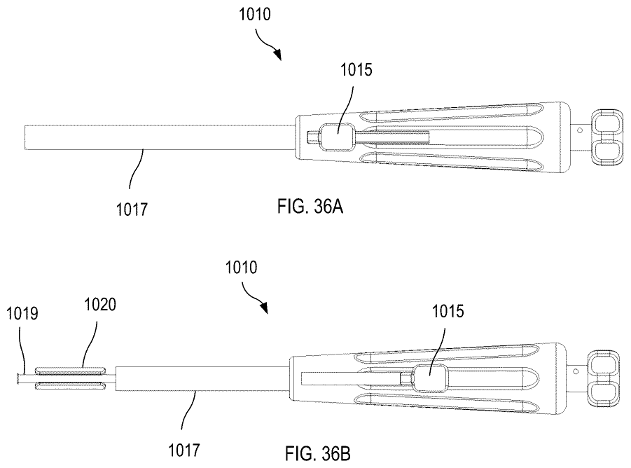

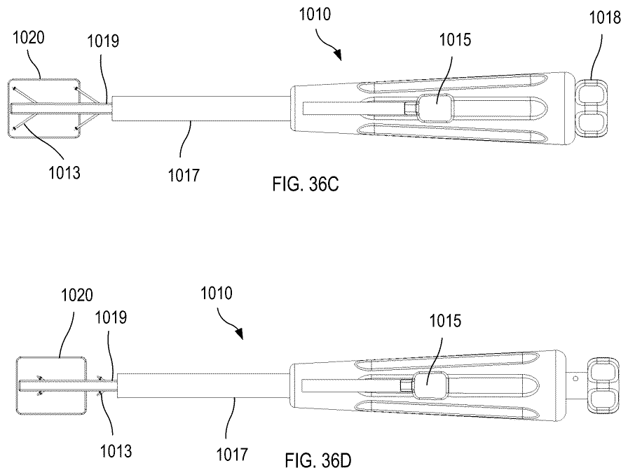

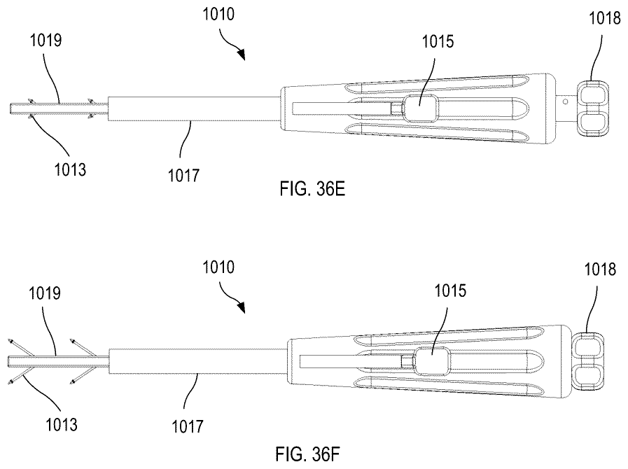

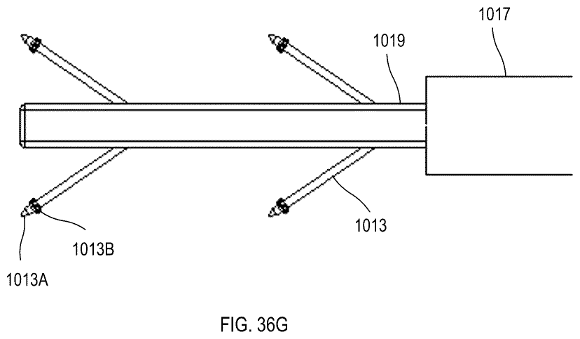

FIGS. 36A to 36G show various aspects of a how a graft deployment device 1010 may be used to deploy a graft 1020 during a graft deployment procedure or method. FIG. 36A depicts the graft deployment device 1010 in an initial configuration (e.g. sheath 1017 extended proximally to cover the graft). In FIG. 36B, the sheath lever 1015 and sheath 1017 have been retracted proximally, and the graft 1020 can be seen loaded on the shaft 1019. As shown in FIG. 36C, the knob 1018 can be advanced distally, thereby advancing the wires 1013 distally through the shaft 1019 and which in turn causes expansion of the graft 1020. As shown in FIG. 36D, once the graft 1020 is fixated the knob 1018 can be retracted proximally, thus retracting the wires 1013. The deployment device 1010 can then be removed, leaving the graft 1010 in place as desired, for example at the patient treatment site. Handle 1016 of the device supports the graft deployment control 1018, which may be a lever, rotatable knob, slider, switch, or other control. The distal end of the device 1010 carries a graft 1020 which can be in a low profile (e.g. initial) configuration for advancement to the treatment site, and as shown in FIGS. 36C and 36D can be in an expanded configuration for implantation and attachment of the graft at the treatment site. Hence the graft 1020 can be expanded from a rolled or folded low profile configuration to a substantially planar expanded configuration. FIGS. 36E and 36F depict actuation of the wires 1013 via distal advancement of the knob 1018 (for purposes of illustration, the graft which is attached with the wires is not shown). As shown in FIG. 36G, a wire 1013 can include a distal tip 1013A configured to puncture a graft. The wire 1013 can also include a stop or widened portion 1013B proximal to the distal tip 1013A. In use, the stop 1013B can prevent the wire 1013 from pushing fully through a graft, for example when the distal tip punctures the graft.

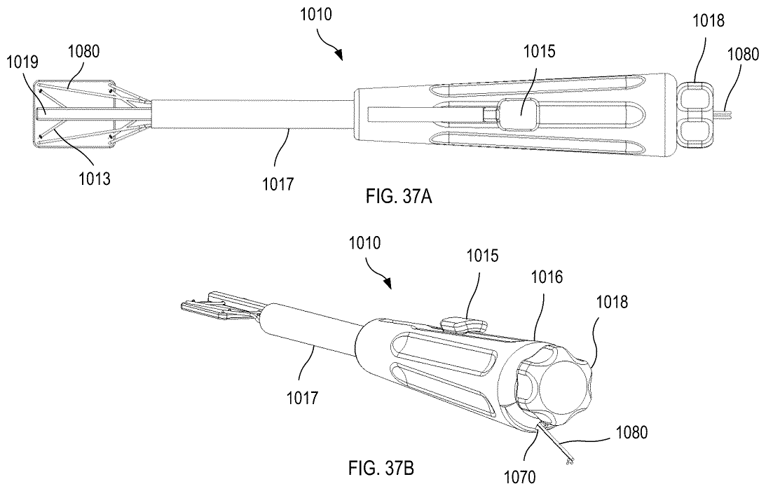

As shown in FIGS. 37A and 37B, a device 1010 can include a suture routing feature or mechanism 1070 (e.g. a channel in the handle 1016 or body) which operates to accommodate a graft 1020 prepared with a suture or suture assembly 1080. In this embodiment, the suture or suture assembly 1080 is coupled with the graft 1020. In some cases, suture routing mechanism 1070 extends through the handle and sheath. The suture routing mechanism can provide a path or channel configured to receive suture therethrough.

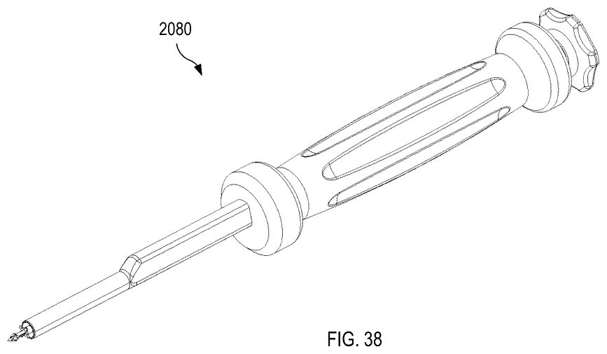

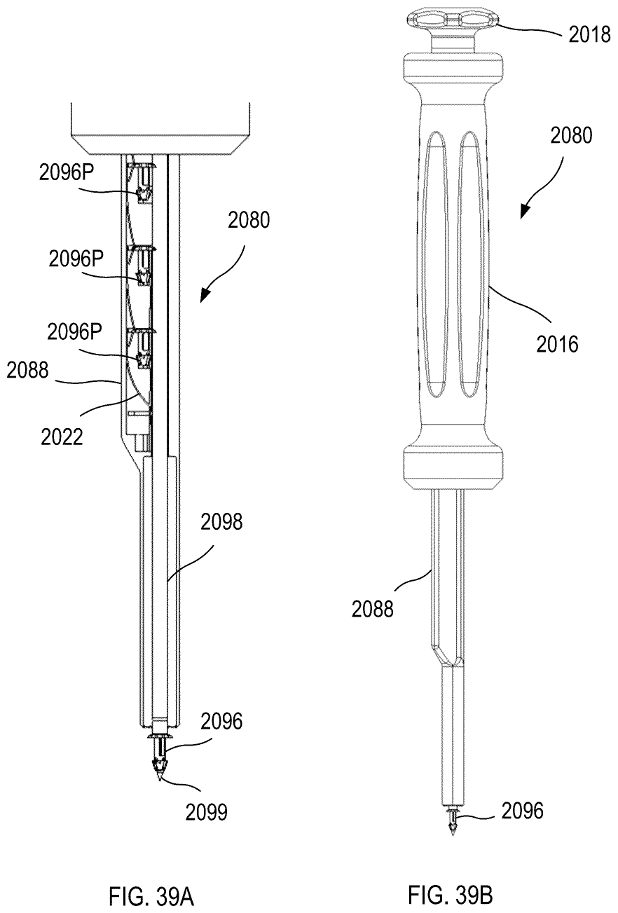

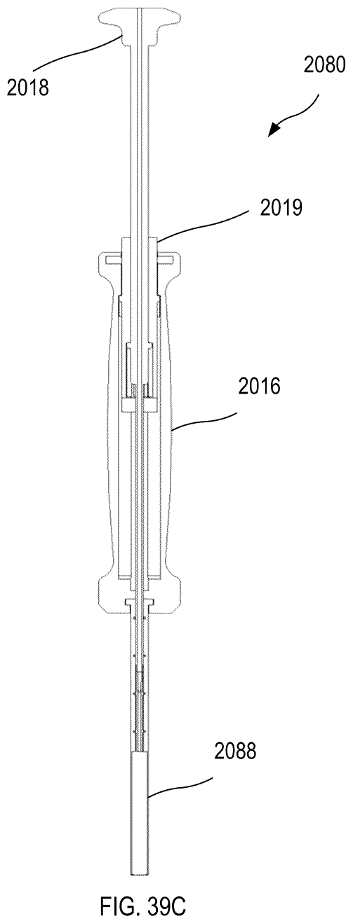

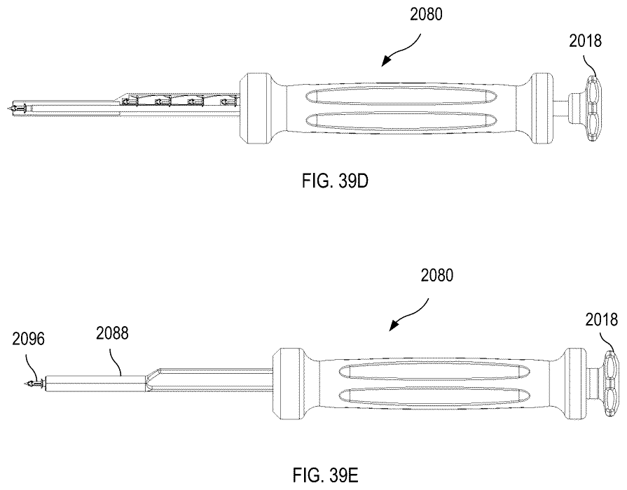

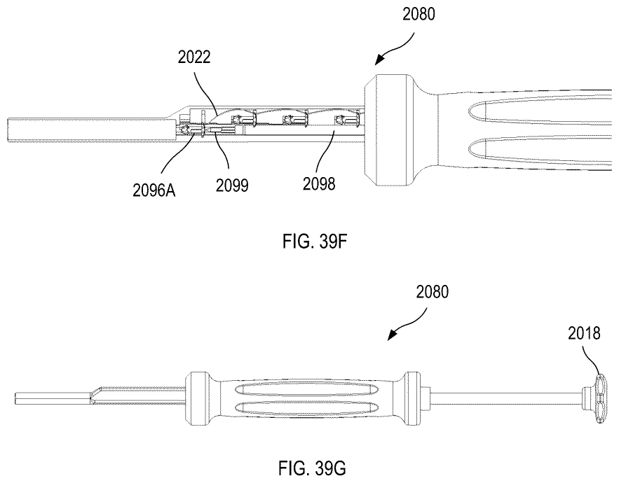

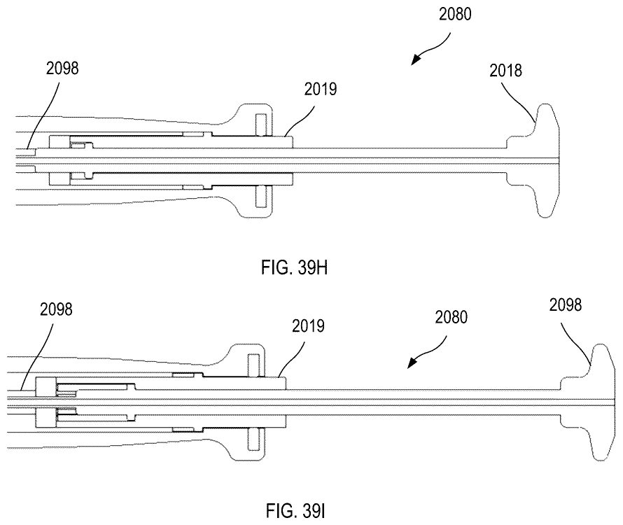

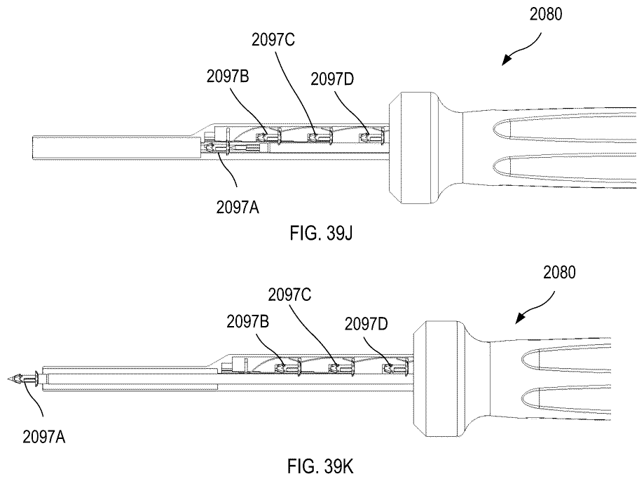

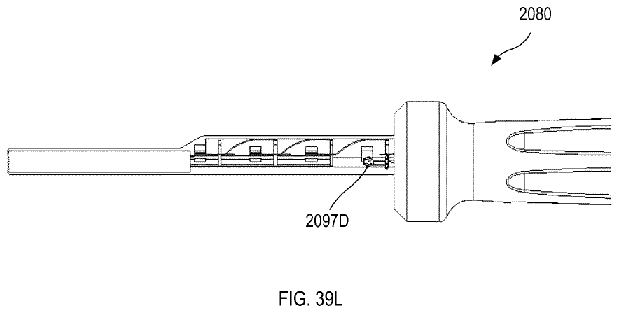

FIG. 38 depicts aspects of a soft tissue fixation deployment device 2080, which in some embodiments may be referred to as an anchor deployment device, a graft fixation deployment device, or a deployment device. As shown in FIGS. 39A and 39B, a fixation deployment device 2080 can include an insertion actuating feature or mechanism 2018, a handle 2016, and a plurality of pre-loaded graft fixation devices (e.g., tacks or anchors) 2096P. In some cases, the fixation devices 2096P can be medial graft fixation devices. As shown here, fixation devices 2096P can be contained within the handle or outer sheath 2088, such as within a cartridge or within a tack or anchor lumen. In the illustrated embodiment, the pre-loaded fixation devices 2096P are coaxially aligned and configured for sequential distal advance into a reloading feature or mechanism. Actuation of a reloading control (e.g., a button or trigger) advances a pre-loaded fixation device 2096P into a deployment ready position such as within the insertion shaft 2098 from which it may be distally advanced through the tack or anchor deployment aperture, such as by distal, axial advance of an awl 2099. Following deployment of a given fixation device 2096, the awl may be proximally retracted and the reloading feature or mechanism 2022 (e.g. a spring) actuated to advance a second pre-loaded fixation device 2096P into a ready position within the cannulated insertion shaft 2098 distally of the awl 2099. The awl 2099 can then be advanced distally to extend the fixation device into a deployment position (e.g. as illustrated by fixation device 2096). In some cases, a fixation device 2096 can include an engagement feature or mechanism, which can operate to engage with the awl 2099.

As shown in FIG. 39C, a fixation deployment device 2080 can include an insertion actuating feature or mechanism 2018, a handle 2016, a stop 2019, and an outer sheath 2088. FIG. 39D depicts fixation deployment device 2080 in an initial configuration. As shown in FIG. 39E, a first dart or fixation device 2096 can be placed or extended from the outer sheath by depressing the insertion actuating feature or mechanism 2018, which may be a plunger, so that the insertion actuating mechanism 2018 is advanced in a distal direction. As shown in FIGS. 39F and 39G, the insertion actuating mechanism 2018 can be drawn back or retracted in a proximal direction, for example until a hard stop is reached. A reloading mechanism or spring 2022 can move the next fixation device 2096A into the path of the shaft 2098, and hence the fixation device 2096A can be operatively engaged with the driver, with the awl 2099 passing through. Advancement of the shaft 2098 (e.g. by moving the shaft 2098 in the distal direction) can operate to place the next fixation device 2096A.

As illustrated in FIGS. 39H and 391, in some embodiments when the shaft 2098 is advanced, the stop 2019 is moved, changing the distance that the shaft can be drawn back to the next reload.

As shown in FIGS. 39J and 39K, one or more of the steps described above can be repeated until all desired anchors are placed. For example, FIG. 39J illustrates how first anchor 2097A can be loaded for placement while second anchor 2097B, third anchor 2097C, and fourth anchor 2097D remain in a pre-loaded configuration. As depicted in FIG. 39K, first anchor 2097A can then be extended distally from the fixation deployment device 2080. FIG. 39L depicts the last remaining anchor, fourth anchor 2097D, disposed in the device after placement of the preceding three anchors.

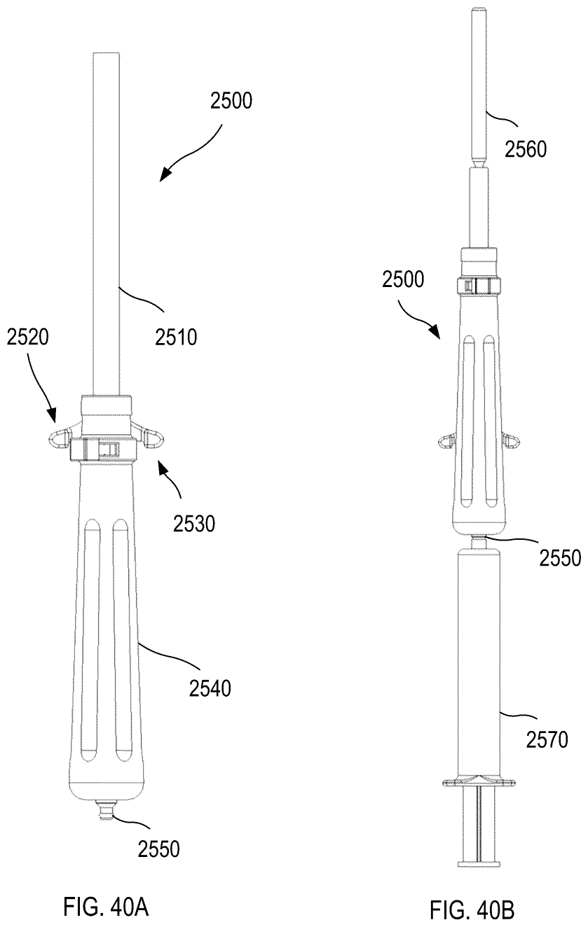

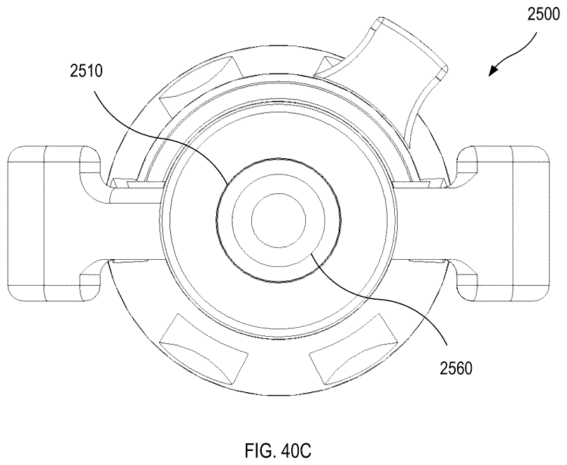

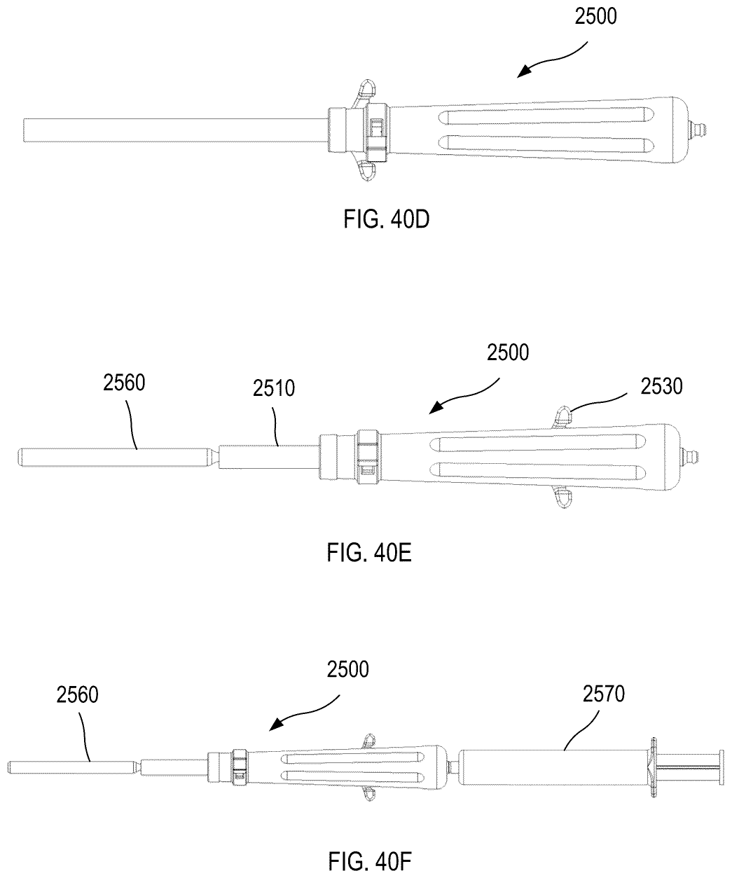

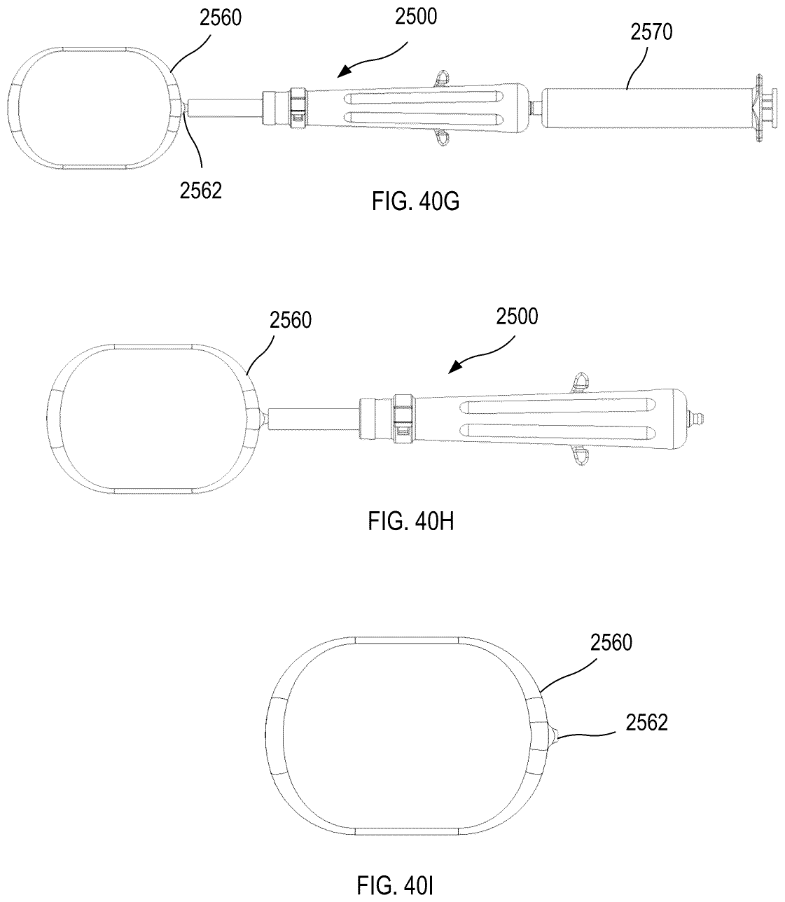

FIGS. 40A to 40I depicts aspects of spacer deployment devices and related methods, according to embodiments of the present invention. As shown in FIG. 40A, a spacer deployment device 2500 can include a sheath 2510, sheath lock 2520, sheath handle 2530, handle 2540, and an inlet 2550 (e.g. fluid inlet, such as a gel inlet). As shown in FIG. 40B, a spacer 2560 can be loaded on the device 2500, and a fluid source 2570, such as a syringe containing a fluid such as a gel, can be coupled in fluid communication with the inlet 2550. As shown in the distal end view provided in FIG. 40C, the spacer deployment device 2500 can include a sheath 2510 and can be loaded with a spacer 2560. FIG. 40D depicts a spacer deployment device 2500 in an initial configuration. As shown in FIG. 40E, once positioned, the sheath handle 2530 can be used to retract the sheath 2510 proximally, thus uncovering or exposing the spacer 2560. As shown in FIG. 40F, a fluid source 2570 can be coupled with the device 2500. In some cases, the fluid source 2570 can be a syringe containing an amount of hydrogel, which can be injected into the spacer 2560. As shown in FIG. 40G, the fluid can be delivered or injected from the fluid source 2570 through the deployment device 2500 and into the spacer 2560, thus expanding the spacer to an expanded configuration. The spacer 2560 can include an inlet 2562 which is configured to receive fluid delivered through the device. The device 2500 can include a lumen or passageway (not shown) through which the fluid is delivered from the fluid source to the spacer. As shown in FIG. 40H, the fluid source can be removed or uncoupled from the device 2500. As shown in FIG. 40I, the device can be removed or uncoupled from the expanded spacer 2560. The spacer 2560 can include an inlet 2562 which is configured to receive fluid delivered through the device.

FIGS. 41A to 41C depict aspects of a loading block 3100, according to embodiments of the present invention. In some embodiments, loading block 3100 can be a graft loading block for loading a graft onto a graft deployment device, for example onto a cylinder or shaft of a graft deployment device. In some cases, loading block 3100 can be referred to as a graft loading block assembly or device or a loading block assembly or device. As shown in FIG. 41A, loading block 3100 can include one or more side needle holes or apertures 3110. As shown in FIG. 41B, loading block 3100 can include one or more top holes 3120, and one or more needle caps, such as a first needle cap 3130 and a second needle cap 3140. As shown in FIG. 41C, a deployment device 3200 can be engaged with the loading block 3100. Needle cap 3130 can attach with needle 3300. In some embodiments, needle 3300 is in operative association with deployment device 3200. For example, deployment device 3200 may include a hole, an aperture, or some other connecting mechanism for engaging the needle. In this way, a distal section of the deployment device 3200 can be attached with a proximal section of the needle 3300. The needle 3300 can be sued to facilitate stitching a suture in a pattern through a graft (e.g. as discussed elsewhere herein with reference to FIGS. 20A to 23H) using the loading block 3100. In some embodiments, the pattern is a sinusoidal pattern. In some embodiments, the pattern approximates a wave such as a sinusoidal wave. In some embodiments, the pattern is a curving pattern. According to some embodiments, a needle cap can be removably attached with a distal end of a needle, for example once the needle is advanced fully through the loading block, in order to prevent the needle from stabbing the user.

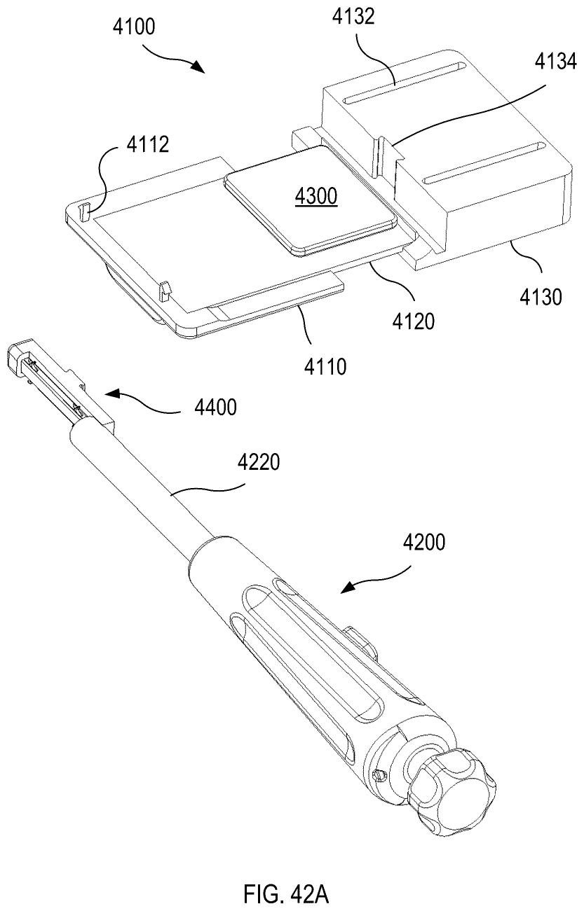

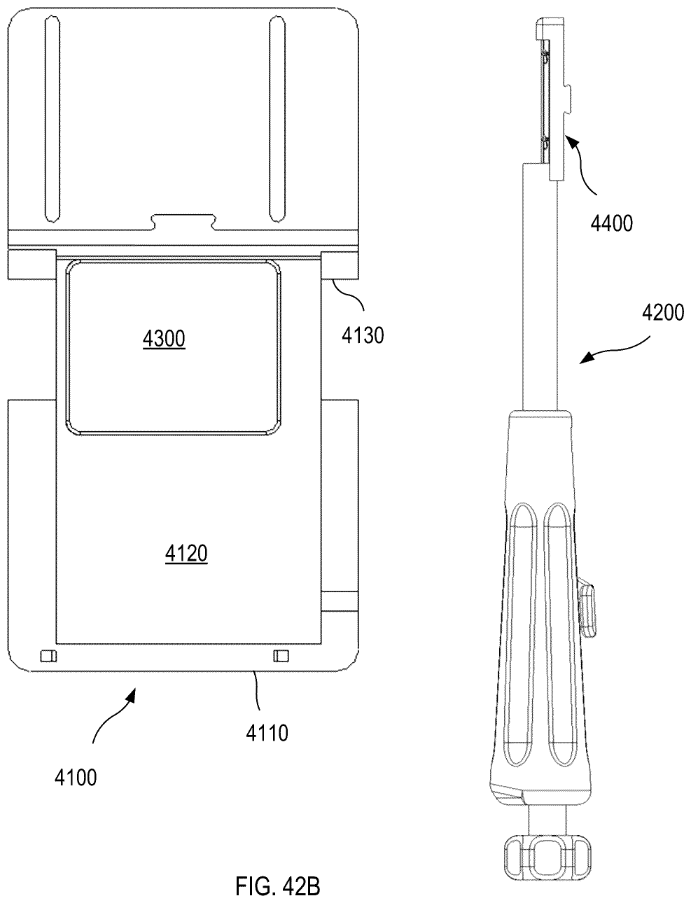

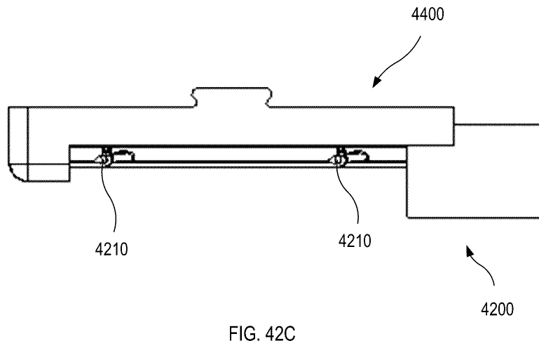

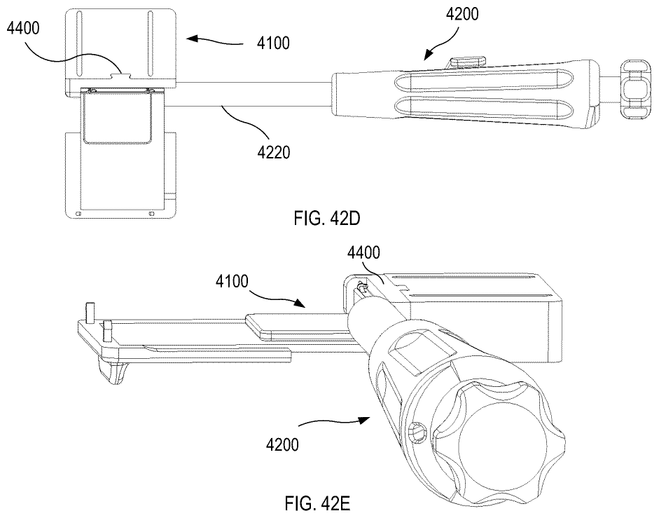

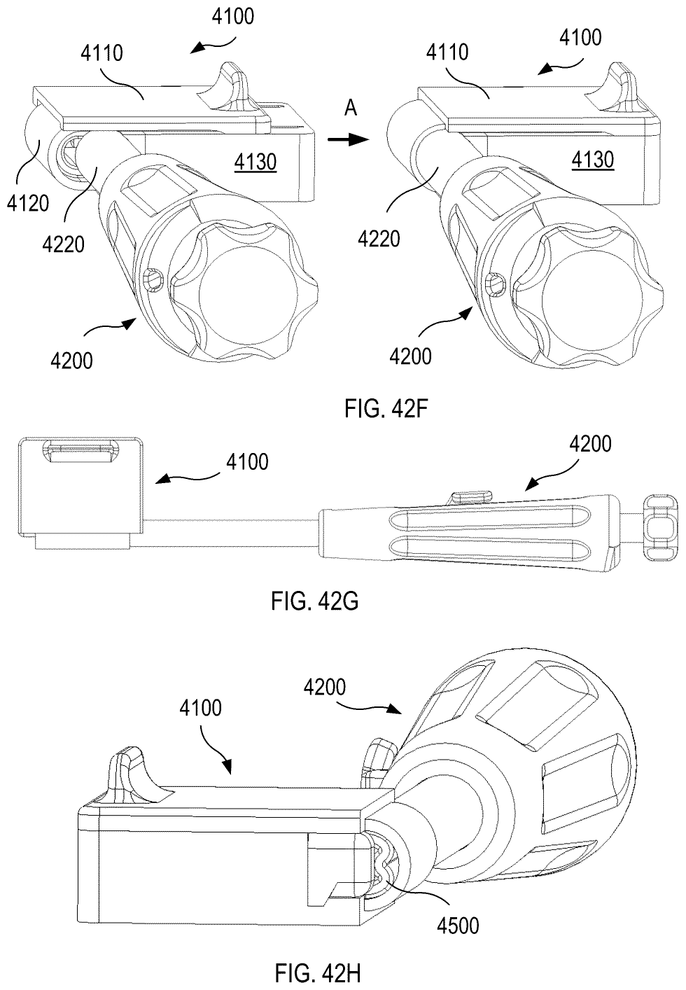

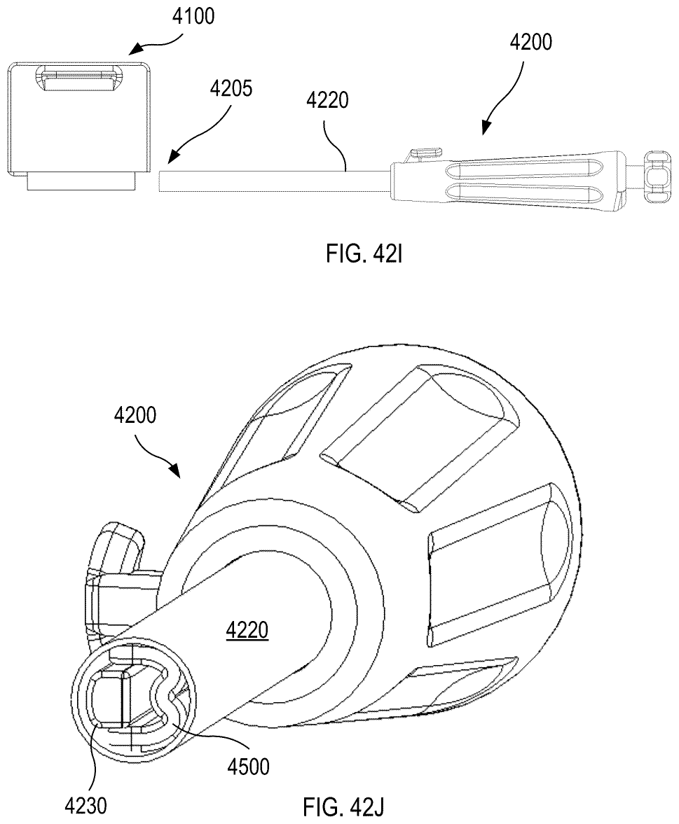

FIGS. 42A to 42J depict aspects of loading blocks, deployment devices, and related graft loading procedures, according to embodiments of the present invention. As shown in FIG. 42A, a graft loading block 4100 can include an upper body 4110 having an upper body locking feature or mechanism 4112, a flexible component 4120, and a lower body 4130 having a lower body locking feature or mechanism 4132. Locking mechanisms 4112 and 4132 can be configured to lock with one another, and when doing so can lock the upper and lower bodies 4110, 4130 together. In some embodiments, upper body 4110 can be a rigid body component, and lower body 4130 can be a rigid body component. In some embodiments, flexible component 4120 can be a flexible and low friction joining component. In some embodiments, the lower body 4130 includes an adapter docking mechanism 4134 that is configured to engage or attach with an adapter 4400. Flexible component 4120 can operate to upper and lower bodies 4110, 4130 to move relative to one another (e.g. as depicted in FIG. 42F). A graft 4300 can be loaded on the loading block 4100. A deployment device 4200 can include or be coupled with a deployment adapter 4400 and can include a shaft 4220. In some embodiments, the adapter 4400 can be loaded on the deployment device 4200.

As shown in FIG. 42B, a graft 4300 can be placed on the loading block 4100 and the adapter 4400 can be attached with the deployment device 4200. In some cases, the adapter 4400 can be provided as a preplaced component of the deployment device. As shown in FIG. 42C, sharpened ends of the wires can be exposed on the graft deployment device 4200. When the deployment adapter 4400 is used to join the deployment device 4200 with the loading block (not shown), the wire ends 4210 can puncture into the graft, fixating a first side of the graft. FIGS. 42D and 42E show the deployment device 4200 and loading block 4100 in operative engagement with one another (e.g. via adapter 4400). In some embodiments, the adapter 4400 can be temporarily placed on the deployment device 4200, and can operate to attach the deployment device 4200 to the loading block 4100 during loading of the graft 4500.

As illustrated in FIG. 42F, the upper body 4110 of the loading block 4100 can be moved or pushed to the side (e.g. transforming the loading block from a first configuration to a second configuration as indicated by arrow A) so as to cause the graft to curl or fold into the size required to fit within the sheath 4220. In this way, the upper and lower bodies can operate or be used to fold or curl the graft. In some cases, this sliding motion can be achieved by cooperative engagement between the locking mechanism of the upper body 4110 and the locking mechanism of the lower body 4130. Hence, in some cases, the locking mechanism of the upper body can be configured for slidable engagement with locking mechanism of the lower body. In some cases, the slidable engagement can be locked or lockable sliding engagement, such that the upper and lower bodies can remain locked together while they slide relative to one another. The folding process may involve positioning the graft in a gap bounded by the upper and lower bodies 4110, 4130 and the flexible component 4120. The bodies 4110, 4130 can be moved relative to each other to cause the joining or flexible component 4120 to constrict the graft, reducing the size of the gap and leaving the graft in a compressed state for loading onto the deployment device 4200. FIGS. 42G and 42H further depict cooperative engagement between loading block 4100 and deployment device 4200, whereby loading block 4100 is used to curl or fold the graft 4500. In some embodiments, based on the size of the graft, the upper body 4110 of the loading block 4100 can be folded over or otherwise moved such that one or more wires of the deployment device 4200 engage into the graft 4500. As shown in FIGS. 411 and 42J, the sheath 4220 can be advanced over the graft 4500, thus pushing the graft loading block 4100 of the distal end 4205 of the deployment device 4200. In this way, the graft 4500 can be loaded on the deployment device 4200. For example, the graft 4500 can be loaded on a shaft or cylinder 4230 of the deployment device 4200. In some embodiments, instead of being loaded into a sheath of a deployment device, the graft 4500 can be loaded into a cannular for delivery to a treatment site of a patient.

Any of the devices or methods disclosed herein can incorporate one or more aspects of any systems, devices, and/or methods disclosed in U.S. patent application Ser. No. 18/382,899 filed Oct. 23, 2023, U.S. patent application Ser. No. 18/678,223 filed May 30, 2024, or U.S. patent application Ser. No. 18/679,041 filed May 30, 2024. The content of each of these three applications is incorporated herein by reference.

Although the preceding description contains significant detail in relation to certain preferred embodiments, it should not be construed as limiting the scope of the invention but rather as providing illustrations of the preferred embodiments.

Embodiments of the present invention encompass kits having one or more components of a system as disclosed herein. In some embodiments, the kit includes one or more system components, along with instructions for using the component(s) for example according to any of the methods disclosed herein.

All features of the described systems and devices described above and in Appendix A are applicable to the described methods mutatis mutandis, and vice versa.

While preferred embodiments of the present disclosure have been shown and described herein, it will be understood to those skilled in the art that such embodiments are provided by way of example only. Numerous variations, changes, and substitutions will now occur to those skilled in the art without departing from embodiments of the present invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A suture loading block device for loading a graft with a suture, comprising:
a top body having a first contoured surface;
a bottom body coupled with the top body, the bottom body having a second contoured surface that is complementary to the first contoured surface of the top body; and
a locking mechanism configured to lock the top body and the bottom body together when the loading block is in a closed configuration,
wherein the first contoured surface and the second contoured surface define a graft space that is configured to receive the graft therein when the loading block device is in the closed configuration,
wherein the first contoured surface comprises a plurality of first ridge troughs,
wherein the second contoured surface comprises a plurality of second ridge troughs, and
wherein the plurality of first ridge troughs and the plurality of second ridge troughs provide a passageway that is configured to receive a needle loaded with the suture therethrough when the loading block device is in the closed configuration.

2. The suture loading block device according to claim 1, wherein the bottom body comprises a cannula that is configured to be in coaxial alignment with the passageway when the suture loading block device is in the closed configuration.

3. The suture loading block device according to claim 1, wherein the first contoured surface comprises a series of first ridges and a series of first valleys, and wherein the second contoured surface comprises a series of second ridges and a series of second valleys.

4. The suture loading block device according to claim 1, wherein the graft space has a sinusoidal configuration.

5. The suture loading block device according to claim 1, wherein the first contoured surface comprises a plurality of third ridge troughs, wherein the second contoured surface comprises a plurality of fourth ridge troughs, and wherein the plurality of third ridge troughs and the plurality of fourth ridge troughs provide a second passageway that is configured to receive a second needle loaded with a second suture therethrough when the suture loading block device is in the closed configuration.

6. The suture loading block device according to claim 1, wherein the top body comprises a top transverse aperture and the bottom body comprises a bottom transverse aperture, and wherein the top transverse aperture and the bottom transverse aperture provide a transverse passageway that is oriented perpendicular to the graft space.

7. The suture loading block device according to claim 1, wherein the needle is attached with a removable handle.

8. The suture loading block device according to claim 1, wherein the needle is attached with a needle passer.

9. The suture loading block device according to claim 1, further comprising a needle cap configured for removeable attachment with a distal end of the needle.

10. The suture loading block device according to claim 1, wherein the locking mechanism comprises a lever.

11. The suture loading block device according to claim 1, wherein the locking mechanism is configured for automatic engagement with a spring load when the suture loading block device is in the closed configuration, and wherein the locking mechanism is configured to be released by actuation of a button.

12. The suture loading block device according to claim 1, wherein the suture loading block device is spring loaded to open when the locking mechanism is released.

13. A method of loading a graft with a suture, the method comprising:
placing the graft on a bottom body of a suture loading block device;
closing a top body of the suture loading block device onto the bottom body, so that the suture loading block device is in a closed configuration and the graft is disposed in a graft space defined by the bottom body and the top body; and
passing a needle loaded with the graft through a passageway defined by the top body and the bottom body, so that the suture is loaded on the graft,
wherein the top body has a first contoured surface, the bottom body has a second contoured surface that is complementary to the first contoured surface of the top body, and wherein the first contoured surface and the second contoured surface define the graft space that is configured to receive the graft therein when the suture loading block device is in the closed configuration, and
wherein the first contoured surface comprises a plurality of first ridge troughs, the second contoured surface comprises a plurality of second ridge troughs, and wherein the plurality of first ridge troughs and the plurality of second ridge troughs provide the passageway.

14. The method according to claim 13, wherein the bottom body comprises a cannula that is configured to be in coaxial alignment with the passageway when the suture loading block device is in the closed configuration.

15. The method according to claim 13, wherein the first contoured surface comprises a series of first ridges and a series of first valleys, and wherein the second contoured surface comprises a series of second ridges and a series of second valleys.

16. The method according to claim 13, wherein the graft space has a sinusoidal configuration.

17. The method according to claim 13, wherein the first contoured surface comprises a plurality of third ridge troughs, wherein the second contoured surface comprises a plurality of fourth ridge troughs, and wherein the plurality of third ridge troughs and the plurality of fourth ridge troughs provide a second passageway that is configured to receive a second needle loaded with a second suture therethrough when the loading block is in the closed configuration.

18. The method according to claim 13, wherein the top body comprises a top transverse aperture and the bottom body comprises a bottom transverse aperture, and wherein the top transverse aperture and the bottom transverse aperture provide a transverse passageway that is oriented perpendicular to the graft space.

19. The method according to claim 13, comprising attaching the needle with a removable handle or a needle passer.

20. The method according to claim 13, comprising removably attaching a needle cap to a distal end of the needle.
